(19) United States
(12) Patent Application Publication
(10) Pub. No.: US 2024/0215881 A1
(43) Pub. Date: Jul. 4, 2024

(54) Identifying and Segmenting Triggering Content

(71) Applicant: Comcast Cable Communications, LLC, Philadelphia, PA (US)

(72) Inventors: Daisy Henderson, Centennial, CO (US); Audite Talukder, Brooklyn, NY (US)

(21) Appl. No.: 18/149,976

(22) Filed: Jan. 4, 2023

(51) Int. Cl. A61B 5/16

(52) U.S. Cl. CPC A61B 5/165

(57) ABSTRACT

Systems, apparatuses, and methods are described for identifying and remediating triggering events in content. Triggering events may be detected via analysis of video, audio, text, and/or metadata components of content segments. Remedial actions based on identified triggers may be offered to users, which may decrease stress symptoms associated with viewing and/or hearing triggering events in content.

### BACKGROUND

Some individuals who may have experienced certain traumatic events may develop stress symptoms, post-traumatic stress disorder (PTSD), and/or other PTSD-type symptoms. Other individuals may have naturally occurring stress symptoms, or may simple wish to avoid certain types triggers or stimuli. The umbrella of individuals affected by such conditions may include war veterans, survivors of natural disasters, survivor of terror attacks, and/or survivors of domestic abuse, among others. These and other individuals may experience symptoms such as depression, anxiety, panic attacks, and/or suicidality. A variety of stimuli reminiscent of the original trauma (which may be referred to as “triggers”, “stress triggers”, etc.) may induce or increase symptoms in those who suffer from these disorders. Various content types (e.g., movies, television programs, advertisements, etc.) may contain triggers which, while innocuous for some audiences, may cause increased stress in those living with stress or disorders, such as PTSD. Triggers may be present in one or more portions of content items, including video, audio, closed captions, and/or metadata.

### SUMMARY

The following summary presents a simplified summary of certain features. The summary is not an extensive overview and is not intended to identify key or critical elements.

Systems, apparatuses, and methods are described for identifying, segmenting, and remediating triggering events in content. Users may denote categories of content which they may find triggering (e.g. stimuli and/or events which may trigger emotional responses, stress, anxiety, PTSD, and/or PTSD-type symptoms). Video data, lighting profile data, audio data, text data, and/or metadata corresponding to content may be analyzed in comparison with databases comprising lists of triggers and/or trigger indicators in order to determine if certain triggers may be present in that content. In cases where triggering events may be identified in content, a variety of remedial actions may be taken which may improve users' content consumption experience by mitigating stress responses to triggers. The remedial actions may comprise skipping the triggering events, summarizing the triggering events, replacing the triggering events with various forms of alternate content, and/or altering the triggering events. A user consuming filtered content (e.g., content in which triggers may have been identified and for which remedial actions may have been presented) may experience fewer symptoms (e.g., anxiety, panic attacks, etc.) which may have been prompted by instances of triggers in appearing in content. Such a user may feel more inclined to consume additional content since filtering may eliminate and/or decrease reactions to triggers in content including movies, television programs, news programs, podcasts, video games, etc.

These and other features and advantages are described in greater detail below.

### BRIEF DESCRIPTION OF THE DRAWINGS

Some features are shown by way of example, and not by limitation, in the accompanying drawings. In the drawings, like numerals reference similar elements.

FIG. 1 shows an example communication network on which the features described herein may be implemented.

FIG. 2 shows hardware elements of a computing device that may be used to implement any of the devices described herein.

FIG. 3 shows an example filtering environment in which the features described herein may be implemented.

FIG. 4 shows a schematic of an example filtering system in which the features described herein may be implemented.

FIGS. 5A, 5B, 5C, and 5D show an example sequence of interfaces for configuring trigger filtering profiles.

FIG. 6 shows an example stress profile interface in which filtering levels may be determined.

FIG. 7 shows an example user account database, upon which trigger filtering may be based.

FIGS. 8A and 8B demonstrate example stress profile databases, in which data corresponding to general and custom stress profiles may be organized, respectively.

FIG. 9A demonstrates an example trigger database in which various types of trigger indicators may be stored.

FIG. 9B illustrates example data indicating characteristics of stress trigger events that have been detected in a filtered content segment.

FIG. 10 is a symbolic drawing showing an example system of devices outputting content items and filtering notifications.

FIG. 11A is an example interface in which a car crash has been identified as a possible stress trigger for the user viewing the content item.

FIG. 11B is an interface showing an example trigger skipping remedial action.

FIG. 11C is an interface showing an example trigger summarizing remedial action.

FIGS. 11D, 11E, 11F, and 11G are interfaces showing example content replacing remedial actions.

FIGS. 11H, 11I, and 11J are interfaces showing example content altering remedial actions.

FIG. 12 shows an example system for altering manifest files.

FIG. 13 is a drawing showing example trigger filtering feedback interfaces.

FIGS. 14A, 14B, and 14C are drawings showing example trigger flagging options in which a user may mark a portion of a content item as containing a triggering event.

FIG. 15A is an example interface illustrating a listing for video-on-demand (VOD) content and options for viewing the content using different trigger filtering profiles.

FIG. 15B is an example interface illustrating a program listing and an option for viewing multicast content with trigger filtering enabled.

FIG. 15C is an example interface for filtering live content and an option for using a buffer delay to allow filtering of live content.

FIG. 16 is an example interface showing content items with indications of their filtered status and options to initiate filtering, view the content, etc.

FIGS. 17A and 17B are flow charts showing an example method for configuring user profiles based on triggers and filtering levels.

FIG. 18 is a flow chart showing an example process for identifying trigger events in a content item.

FIGS. 19A, 19B, 19C, and 19D are flow charts showing example methods for implementing various remedial actions based on triggering events in content.

FIGS. 20A and 20B are flow charts showing example operational methods for presenting and remediating content to a user device.

### DETAILED DESCRIPTION

The accompanying drawings, which form a part hereof, show examples of the disclosure. It is to be understood that the examples shown in the drawings and/or discussed herein are non-exclusive and that there are other examples of how the disclosure may be practiced.

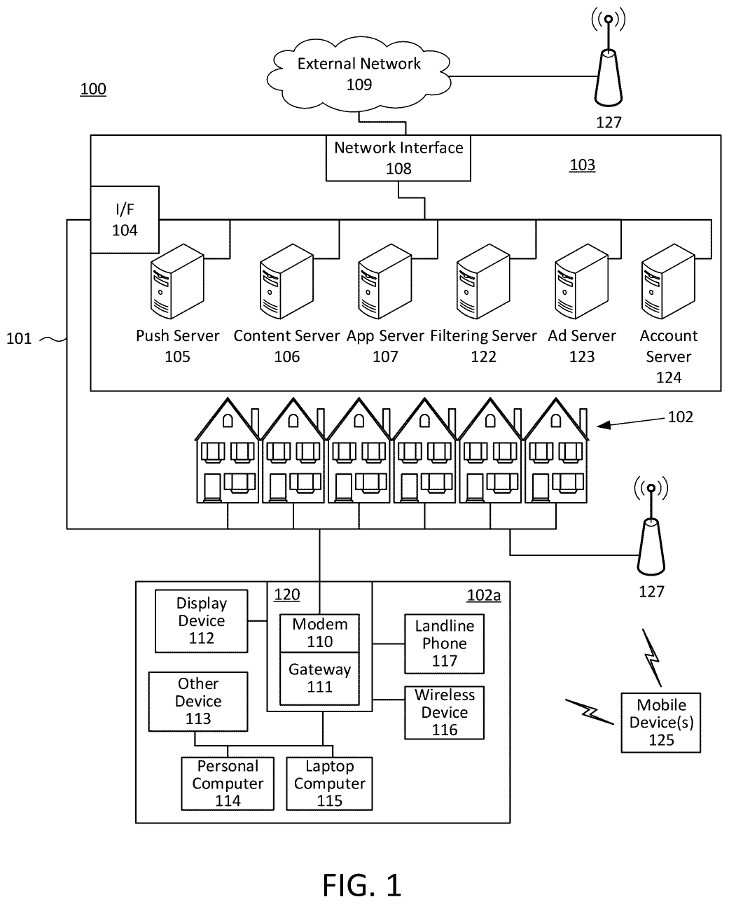

FIG. 1 shows an example communication network 100 in which features described herein may be implemented. The communication network 100 may comprise one or more information distribution networks of any type, such as, without limitation, a telephone network, a wireless network (e.g., an LTE network, a 5G network, a WiFi IEEE 802.11 network, a WiMAX network, a satellite network, and/or any other network for wireless communication), an optical fiber network, a coaxial cable network, and/or a hybrid fiber/coax distribution network. The communication network 100 may use a series of interconnected communication links 101 (e.g., coaxial cables, optical fibers, wireless links, etc.) to connect multiple premises 102 (e.g., businesses, homes, consumer dwellings, train stations, airports, etc.) to a local office 103 (e.g., a headend, a central location). The local office 103 may send downstream information signals and receive upstream information signals via the communication links 101. Each of the premises 102 may comprise devices, described below, to receive, send, and/or otherwise process those signals and information contained therein.

The communication links 101 may originate from the local office 103 and may comprise components not shown, such as splitters, filters, amplifiers, etc., to help convey signals clearly. The communication links 101 may be coupled to one or more wireless access points 127 configured to communicate with one or more mobile devices 125 via one or more wireless networks. The mobile devices 125 may comprise smart phones, tablets or laptop computers with wireless transceivers, tablets or laptop computers communicatively coupled to other devices with wireless transceivers, and/or any other type of device configured to communicate via a wireless network.

The local office 103 may comprise an interface 104. The interface 104 may comprise one or more computing devices configured to send information downstream to, and to receive information upstream from, devices communicating with the local office 103 via the communications links 101. The interface 104 may be configured to manage communications among those devices, to manage communications between those devices and backend devices such as servers 105-107 and 122-124 (described below), and/or to manage communications between those devices and one or more external networks 109. The interface 104 may, for example, comprise one or more routers, one or more base stations, one or more optical line terminals (OLTs), one or more termination systems (e.g., a modular cable modem termination system (M-CMTS) or an integrated cable modem termination system (I-CMTS)), one or more digital subscriber line access modules (DSLAMs), and/or any other computing device(s). The local office 103 may comprise one or more network interfaces 108 that comprise circuitry needed to communicate via the external networks 109. The external networks 109 may comprise networks of Internet devices, telephone networks, wireless networks, wired networks, fiber optic networks, and/or any other desired network. The local office 103 may also or alternatively communicate with the mobile devices 125 via the interface 108 and one or more of the external networks 109, e.g., via one or more of the wireless access points 127.

The push notification server 105 may be configured to generate push notifications to deliver information to devices in the premises 102 and/or to the mobile devices 125. The content server 106 may be configured to provide content to devices in the premises 102 and/or to the mobile devices 125. This content may comprise, for example, video, audio, text, web pages, images, files, etc. The content server 106 (or, alternatively, an authentication server) may comprise software to validate user identities and entitlements, to locate and retrieve requested content, and/or to initiate delivery (e.g., streaming) of the content. Examples of content servers 106 may include computing devices that provide streaming Internet content, such as the streaming services offered by PEACOCK, NETFLIX, AMAZON PRIME, HULU, HBO MAX, DISNEY+, NBC, CNN, HBO, etc. The content server 106 may comprise filtered (e.g., preprocessed content) and/or unfiltered content. The content server 106 may include content which may comprise, for example, video (e.g., movies, television programming, etc.), audio, text, web pages, files, images, etc. The application server 107 may be configured to offer any desired service. For example, an application server may be responsible for collecting, and generating a download of, information for electronic program guide listings. Another application server may be responsible for monitoring user viewing habits and collecting information from that monitoring for use in selecting advertisements. Yet another application server may be responsible for formatting and inserting advertisements in a video stream being transmitted to devices in the premises 102 and/or to the mobile devices 125. The local office 103 may comprise additional servers, such as the filtering server 122, the advertisement server 123, the account server 124, additional push, content, and/or application servers, and/or other types of servers.

The filtering server 122 may comprise software for analyzing content. The filtering of content may comprise processing the content to identify potential stress-triggering events, so that they may be dealt with when a user is susceptible to those events. The analyzing may include processing of video data, lighting profile data, audio data, text data, and/or metadata corresponding to content which may be retrieved from the content server 106, the advertisement server 123, and/or other servers. Lighting profile data may be variously retrieved, for example, lighting profile data may be comprised in video data (e.g., it may be retrieved based on analysis of video data) and/or lighting profile data may be comprised in lighting profiles (e.g., which may include indications of lighting features within a content item). Lighting profiles may be comprised in preprocessed content items and/or they may be generated based on analyzing the content items (e.g., via the filtering server 122). Metadata may comprise lighting profile data, which may summarize lighting effects in a content item, such as flashes of light, changes in brightness, etc. For example, lighting profile data may indicate that brightness increases by 80% at timestamp 10:31 in a movie, which may indicate the presence of a triggering event such as a muzzle flash at that time.

Content analysis software may include searching content for events which may trigger adverse emotional reactions, such as PTSD symptoms, in certain users. The analysis software and/or the filtering server 122 may be implemented by one or more machine learning algorithms. The filtering server 122 may comprise software for dynamically inserting breaks and/or other content segments in content to help the users avoid and/or deal with triggering content (e.g., using features of Society of Cable Telecommunications Engineers/ANSI joint standards SCTE-35, SCTE-104, and/or other standards), which may be inserted in a manifest file corresponding to certain content. Manifest files may include, for example, instructions for retrieving content fragments which may be delivered to user devices, various metadata such as closed captioning data and/or content summaries, and/or markers which may be used to dynamically insert breaks, advertisements, alternate scenes, etc. The advertisement server 123 may be configured to offer advertisements. Users may receive advertisements filtered for potentially triggering events based on their preferences. The advertisement server 123 may comprise software for dynamically inserting advertisements in content (e.g., using features of SCTE-35, SCTE-104, and/or other standards).

The account server 124 may be configured to store encrypted and/or otherwise secured user information. For example, the account server 124 may be configured to be a double-blind server, such that encrypted user data (e.g., personal, sensitive, and/or identifying user information) which may be stored on the server 124 may not be accessible in an unsecured format by devices in the local office 103, the external network 109, devices in the premises 102, and/or other devices and/or networks. A user may disclose their preference for handling of triggering events in a user profile account, which may be stored as encrypted data. In serving this user filtered content, an internet service provider, content provider, and/or other entities may not be able to associate the user's status with their identity.

Although shown separately, the push server 105, the content server 106, the application server 107, the filtering server 122, the advertisement server 123, the account server 124, and/or other server(s) may be combined. The servers 105, 106, 107, 122, 123, 124 and/or other servers, may be computing devices and may comprise memory storing data and also storing computer executable instructions that, when executed by one or more processors, cause the server(s) to perform steps described herein.

An example premises 102a may comprise an interface 120. The interface 120 may comprise circuitry used to communicate via the communication links 101. The interface 120 may comprise a modem 110, which may comprise transmitters and receivers used to communicate via the communication links 101 with the local office 103. The modem 110 may comprise, for example, a coaxial cable modem (for coaxial cable lines of the communication links 101), a fiber interface node (for fiber optic lines of the communication links 101), twisted-pair telephone modem, a wireless transceiver, and/or any other desired modem device. One modem is shown in FIG. 1, but a plurality of modems operating in parallel may be implemented within the interface 120. The interface 120 may comprise a gateway 111. The modem 110 may be connected to, or be a part of, the gateway 111. The gateway 111 may be a computing device that communicates with the modem(s) 110 to allow one or more other devices in the premises 102a to communicate with the local office 103 and/or with other devices beyond the local office 103 (e.g., via the local office 103 and the external network(s) 109). The gateway 111 may comprise a set-top box (STB), digital video recorder (DVR), a digital transport adapter (DTA), a computer server, and/or any other desired computing device.

The gateway 111 may also comprise one or more local network interfaces to communicate, via one or more local networks, with devices in the premises 102a. Such devices may comprise, e.g., display devices 112 (e.g., televisions), other devices 113 (e.g., a DVR or STB), personal computers 114, laptop computers 115, wireless devices 116 (e.g., wireless routers, wireless laptops, notebooks, tablets and netbooks, cordless phones (e.g., Digital Enhanced Cordless Telephone-DECT phones), mobile phones, mobile televisions, personal digital assistants (PDA)), landline phones 117 (e.g., Voice over Internet Protocol-VoIP phones), and any other desired devices. Example types of local networks comprise Multimedia Over Coax Alliance (MoCA) networks, Ethernet networks, networks communicating via Universal Serial Bus (USB) interfaces, wireless networks (e.g., IEEE 802.11. IEEE 802.15, Bluetooth), networks communicating via in-premises power lines, and others. The lines connecting the interface 120 with the other devices in the premises 102a may represent wired or wireless connections, as may be appropriate for the type of local network used. One or more of the devices at the premises 102a may be configured to provide wireless communications channels (e.g., IEEE 802.11 channels) to communicate with one or more of the mobile devices 125, which may be on- or off-premises.

The mobile devices 125, one or more of the devices in the premises 102a, and/or other devices may receive, store, output, and/or otherwise use assets. An asset may comprise a video, a game, one or more images, software, audio, text, webpage(s), and/or other content.

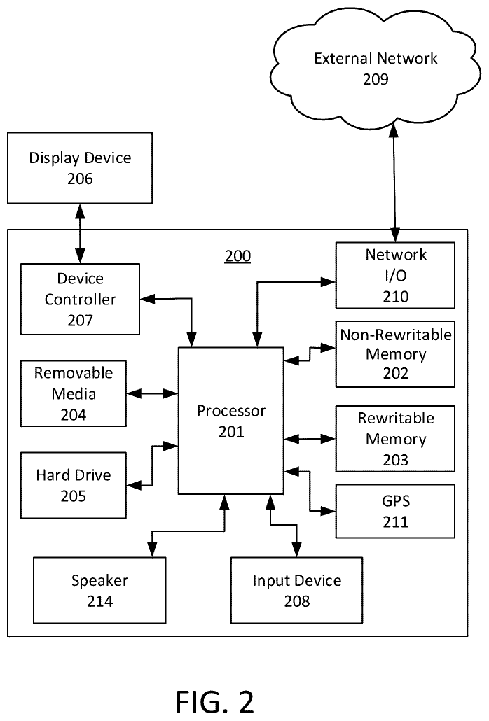

FIG. 2 shows hardware elements of a computing device 200 that may be used to implement any of the computing devices shown in FIG. 1 (e.g., the mobile devices 125, any of the devices shown in the premises 102a, any of the devices shown in the local office 103, any of the wireless access points 127, any devices with the external network 109) and any other computing devices discussed herein. For example, the computing device 200 may be used to implement any of the processes comprised in the filtering server 122. The computing device 200 may comprise one or more processors 201, which may execute instructions of a computer program to perform any of the functions described herein. The instructions may be stored in a non-rewritable memory 202 such as a read-only memory (ROM), a rewritable memory 203 such as random-access memory (RAM) and/or flash memory, removable media 204 (e.g., a USB drive, a compact disk (CD), a digital versatile disk (DVD)), and/or in any other type of computer-readable storage medium or memory. Instructions may also be stored in an attached (or internal) hard drive 205 or other types of storage media. The computing device 200 may comprise one or more output devices, such as a display device 206 (e.g., an external television and/or other external or internal display device) and a speaker 214, and may comprise one or more output device controllers 207, such as a video processor or a controller for an infra-red or BLUETOOTH transceiver. One or more user input devices 208 may comprise a remote control, a keyboard, a mouse, a touch screen (which may be integrated with the display device 206), microphone, etc. The computing device 200 may also comprise one or more network interfaces, such as a network input/output (I/O) interface 210 (e.g., a network card) to communicate with an external network 209. The network I/O interface 210 may be a wired interface (e.g., electrical, RF (via coax), optical (via fiber)), a wireless interface, or a combination of the two. The network I/O interface 210 may comprise a modem configured to communicate via the external network 209. The external network 209 may comprise the communication links 101 discussed above, the external network 109, an in-home network, a network provider's wireless, coaxial, fiber, or hybrid fiber/coaxial distribution system (e.g., a DOCSIS network), or any other desired network. The computing device 200 may comprise a location-detecting device, such as a global positioning system (GPS) microprocessor 211, which may be configured to receive and process global positioning signals and determine, with possible assistance from an external server and antenna, a geographic position of the computing device 200.

Although FIG. 2 shows an example hardware configuration, one or more of the elements of the computing device 200 may be implemented as software or a combination of hardware and software. Modifications may be made to add, remove, combine, divide, etc. components of the computing device 200. Additionally, the elements shown in FIG. 2 may be implemented using basic computing devices and components that have been configured to perform operations such as are described herein. For example, a memory of the computing device 200 may store computer-executable instructions that, when executed by the processor 201 and/or one or more other processors of the computing device 200, cause the computing device 200 to perform one, some, or all of the operations described herein. Such memory and processor(s) may also or alternatively be implemented through one or more Integrated Circuits (ICs). An IC may be, for example, a microprocessor that accesses programming instructions or other data stored in a ROM and/or hardwired into the IC. For example, an IC may comprise an Application Specific Integrated Circuit (ASIC) having gates and/or other logic dedicated to the calculations and other operations described herein. An IC may perform some operations based on execution of programming instructions read from ROM or RAM, with other operations hardwired into gates or other logic. Further, an IC may be configured to output image data to a display buffer.

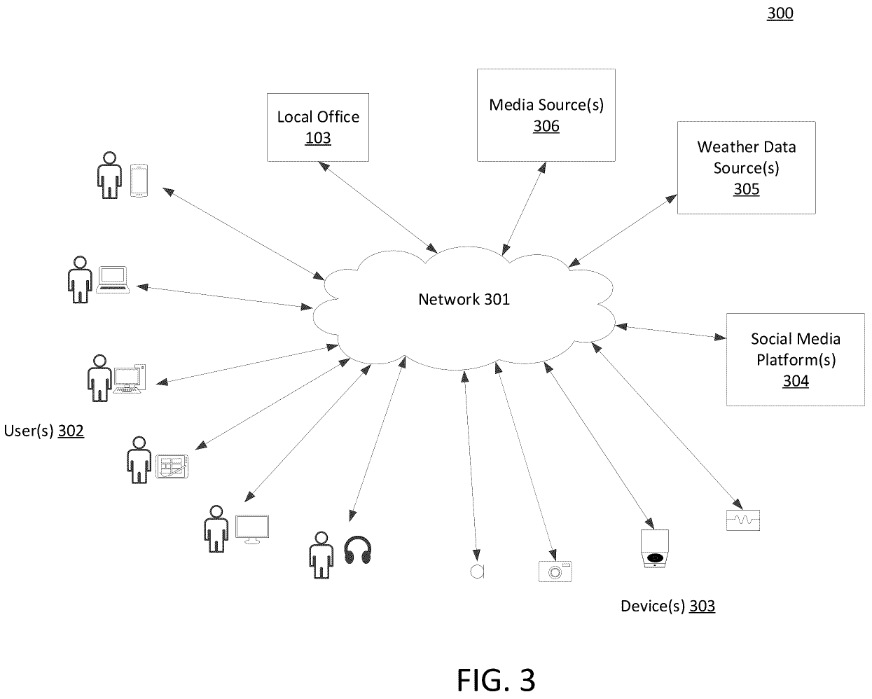

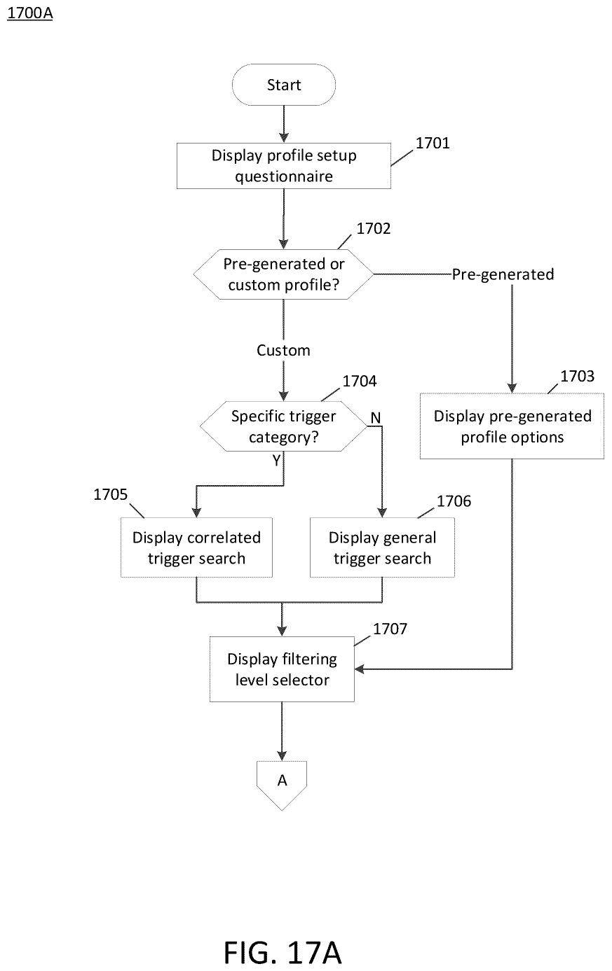

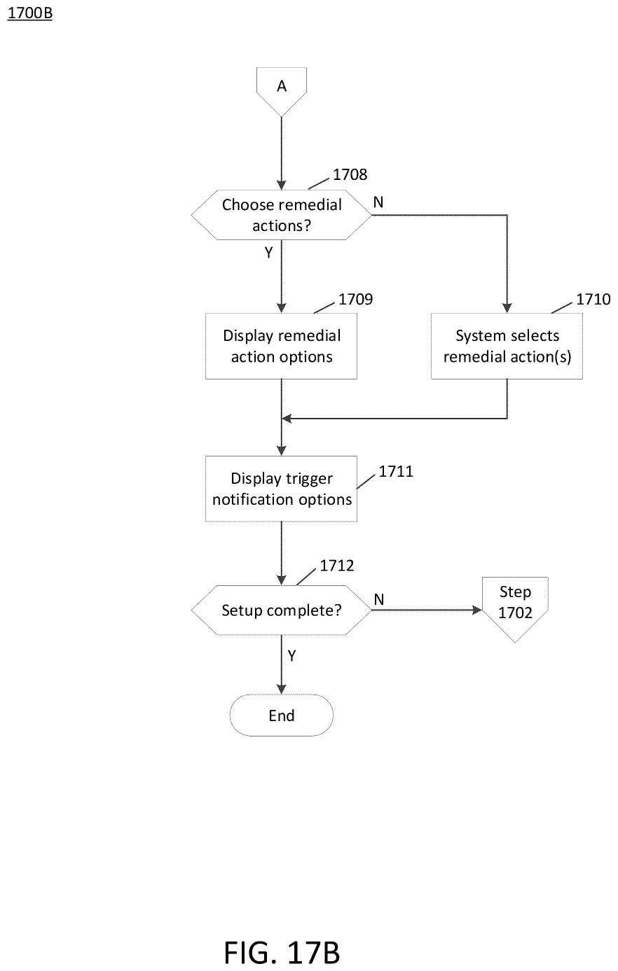

As will be described herein, a user may be vulnerable to certain types of stress events. For example, if a user is a gunshot survivor and has a risk of suffering a stress reaction from seeing gun violence in movies, then features described herein may help the user deal with scenes of gun violence in content that the user watches. Content metadata may indicate when certain types of stress-triggering events are found in a content item, and if a user is vulnerable to an upcoming stress event, then the system may provide the user with an advance warning of the event. The system may provide the user with selectable options to avoid seeing the event, such as by skipping past a shootout scene, and/or by replacing the shootout scene with an alternative scene (e.g., perhaps with the gun sounds and images omitted), or even with a textual description of the scene being skipped (to allow the user to still follow the story of the movie. The use of features described in FIGS. 3-16 will be explained with the algorithms in FIGS. 17-21, described further below.

FIG. 3 shows an example environment 300 in which stress triggering events in content may be handled. The environment 300 may comprise a network 301, which may connect external networks 109, 209, and/or other internal and/or external networks, and may allow various types of devices to communicate with one another. The local office 103 may be connected to the network 301. Users 302, devices 303, social media platforms 304, weather data sources 305, and/or media sources 306 may be connected to the network 301. Components connected to the network 301 may communicate with each other and/or with other components via the network 301. Users 302 may interact with the network 301 and/or other components connected to the network via a variety of devices, which may include: smartphones, personal computing (PC) devices such as laptop and/or desktop computers, portable media consumption devices, tablet computers, displays such as televisions, Internet of Things (IOT) devices, wireless listening devices such as Bluetooth headphones, smart wearable devices such as smart glasses, and/or voice assistants. While the illustration in FIG. 3 demonstrates six examples of users, any number of users may be connected to the network 301 and/or other networks. The users 302 may be connected within the same premises and/or from different premises. References to the users 302 may include their devices.

The devices 303 may include input devices that provide the system with input for understanding and/or managing the user's stress level. The devices 303 may include any type of device that can provide information about a user, such as microphones, cameras, motion sensors, voice assistants, health devices, smart watches, heart rate monitors, skin conductance sensors, noninvasive brain computer interfaces, etc. The devices 303 may transmit user data to various components connected to the network 301, such as the account server 124, which may store users' trigger preferences.

The environment 300 may include data sources including the social media platforms 304 (e.g., FACEBOOK, TWITTER, INSTAGRAM, YOUTUBE, TIKTOK, LINKEDIN), the weather data sources 305 (e.g., the National Weather Service, the National Oceanic and Atmospheric Administration (NOAA), the National Aeronautics and Space Association (NASA), the Environmental Protection Agency), and/or the media sources 306 (e.g., video streams, television channels, on-demand multimedia content, radio stations, newspapers, news networks, media outlets, magazines, social media platforms, etc.). Filtering of content may comprise searching the data sources 304-306 for information indication the presence of triggers in the content (examining metadata for flags indicating pre-identified scenes, image recognition to identify stress-inducting images. Social media platforms 304 may comprise data indicating the presence of triggers in the content (e.g., social media posts by viewers discussing a stress-triggering scene in the film), which may be retrieved during processing of the film (described below).

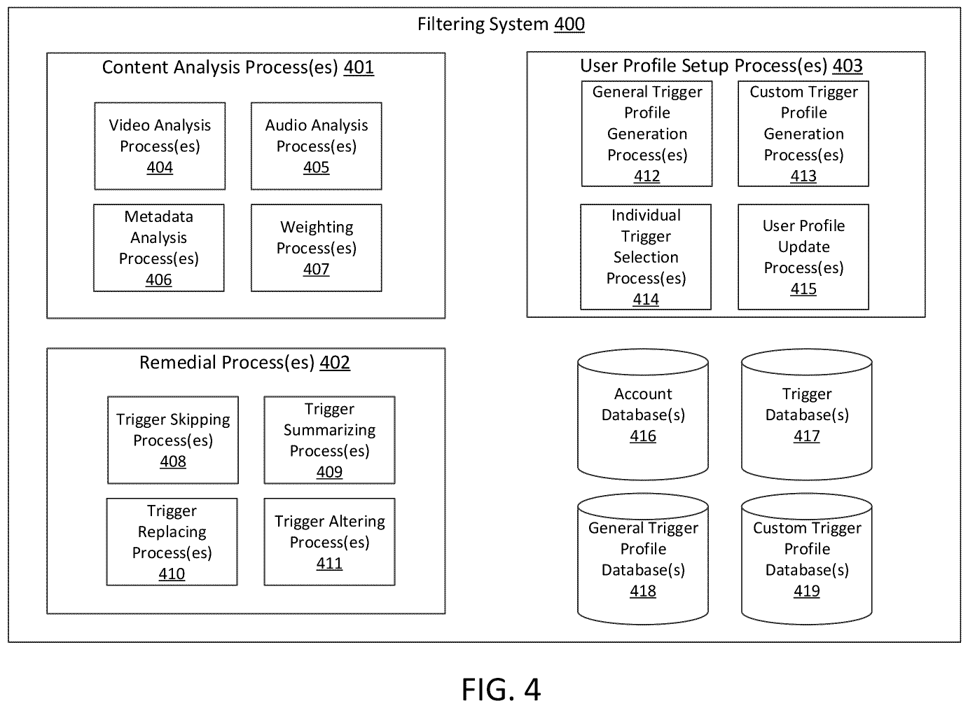

FIG. 4 shows a schematic of an example system 400. The system 400 may comprise content analysis processes 401, remedial processes 402, user profile setup processes 403, account databases 416, trigger databases 417, general stress profile databases 418, and/or custom stress profile databases 419. The various processes and/or databases described herein may be implemented by any device(s) described above and/or machine learning algorithms.

The content analysis processes 401 may analyze content, such as a movie, to generate data indicating the types and/or occurrences of stress-triggering events in the content, which may include lighting profile data. The content analysis processes 401 may be implemented by various devices, for example, they may be implemented by devices in the local office 103, the premises 102a, and/or others. Lighting profile data may be based on video data and/or may be comprised in metadata. Lighting profiles may include information indicating brightness (which may be represented by luminescence values) and/or changes in lighting associated with certain triggers. Lighting profiles may be generated based on image detection performed on video data. For example, for a content item, video data may be analyzed such that the video frames corresponding to a particular segment may be retrieved, pixel values (e.g., color, brightness, luminescence, and/or other lighting values, etc.) may be retrieved, variations in lighting may be retrieved (e.g., between sequential frames), and/or locations of the lighting profile data within the content item may be stored. Lighting profile data may include flashing lights, tones of light, and/or variations in lighting. Lighting profile data may indicate the presence of triggers (e.g., muzzle flashes, explosions, and/or helicopter blades). For example, in an action movie, muzzle flashes may be indicated by lighting profiles showing sudden changes in luminescence, explosions may be indicated by lighting profiles showing increases in overall brightness and cool toned light, and spinning helicopter blades may be indicated by lighting profiles showing rapidly flashing lights.

The content analysis processes 401 may include video analysis processes 404, audio analysis processes 405, metadata analysis processes 406, and/or weighting processes 407; the sub-processes 404-407 will be described below. The video analysis processes 404 may analyze video data associated with the content to identify triggers, for example, via methods for analyzing video data and/or generating lighting profiles. The audio analysis processes 405 may perform the same features as the video analysis processes 404 on audio data associated with the content, including methods for taking in audio data, detecting certain indicators of audio triggers (e.g., sounds of gunfire, explosions, screams, etc.), analyzing different audio tracks for the same segment (e.g., dialogue, sound effects, soundtrack, etc.) separately and/or in combination, etc. The metadata analysis processes 406 may perform the same features as the processes 404-405 on metadata associated with the content, including analyzing metadata to identify text indicators of certain triggers (e.g., phrases, dialogue, individual words, and/or synonyms). Metadata may include any data in manifest files associated with the content, closed captioning data, summaries of content, codes corresponding to triggers, descriptions of content, and/or lighting profiles. The metadata analysis processes 406 may analyze lighting profiles to identify indicators (e.g., visual and/or metadata indicators) of triggers associated with variations in lighting and/or other features comprised in lighting profile data. Lighting profiles may vary and/or overlap for certain triggers (e.g., certain muzzle flashes and explosions may correlate with similar and/or the same lighting profiles, and other indicators such as audio, text, and/or metadata may be used to differentiate the triggers). Identification of triggers may be performed with greater accuracy when considering multiple types of indicators found in video data, lighting profile data, audio data, text data, and/or metadata (e.g., a gunshot may be identified with increased accuracy if an image of a gun, a gunshot sound, and a muzzle flash lighting profile are contemporaneously detected in a content item).

The weighting processes 407 may calculate severities of identified triggers and/or apply markers corresponding to the calculated severities via methods for calculating and/or assigning weights (e.g., indicators of severity, frequency, etc.) to certain triggers and/or trigger indicators. Calculated trigger severities may be determined based on individual user preferences (e.g., user data relating to sensitivity to certain triggers).

The remedial processes 402 may perform remedial actions when triggering events have been identified in content, when triggering events may have been presented to users, and/or when triggering events are about to be presented to users. The remedial processes 402 may include trigger skipping processes 408, trigger summarizing processes 409, trigger replacing processes 410, and/or trigger altering processes 411; the sub-processes 408-411 will be described below. Any of the remedial processes described herein may be applied to the same stress-triggering events, including, for example, simultaneous execution of one or more of the remedial processes 402. The trigger summarizing processes 409 may comprise methods for generating triggering event summaries based on metadata and/or results from searching external sources (e.g., internet encyclopedias, social media platforms, content databases, etc.).

The trigger replacing processes 410 may comprise methods for retrieving and/or inserting alternate content corresponding to triggering events identified in content. Examples of alternate content include alternate content segments and/or scenes (e.g., scenes which may be less triggering and/or more family friendly, which may be rated for general audiences, which may be edited for broadcast and/or multicast, and/or which may retain the plot of the original content while not including certain triggers), content which may be considered calming, remedial, and/or helpful in reducing symptoms such as anxiety (e.g., nature scenes, guided breathing and/or meditation, etc.), counseling resources (e.g., mental health support resources, links to external support resources such as veterans' organizations and/or mental health professionals, etc.), and/or advertisements which may be filtered for triggers according to users' preferences.

A user may choose to have scenes of gun violence replaced with a calming nature scene (e.g., a gentle river) and a textual overlay describing the triggering events being replaced. The trigger altering processes 411 may alter containing triggering events and/or the triggering events themselves via methods including altering video data, audio data, text data, and/or metadata corresponding to triggering content. Altering may include lowering and/or raising volume, silencing audio, altering brightness, altering contrast, blurring at least a portion of the triggering events, and/or obscuring and/or blocking at least a portion of the triggering events. A user whose PTSD symptoms may be triggered by explosions which may be indicated by bright flashes may elect to have brightness and contrast lowered for scenes of explosions, which may allow them to continue watching the content and may mitigate their symptoms.

The user profile setup processes 403 (e.g., stress profile configuration processes) may generate stress trigger filtering profiles including pre-generated profiles based on common categories of PTSD triggers (e.g., war, assault, etc.) useful for many types of users and/or customized profiles tailored to individual users. The user profile setup processes 403 may include general stress profile generation processes 412, custom stress profile generation processes 413, individual trigger selection processes 414, and/or user profile update processes 415. The general stress profile generation processes 412 may include methods for generating stress profiles that may be applicable to a wide range of users who may experience common sets of triggers, which may allow users identifying with certain general trigger categories to filter content using a general profile. The general profiles may indicate certain remedial actions (e.g., resulting from any one or more of the user profile setup processes 403) for triggers. The custom stress profile generation processes 413 may include methods for generating stress profiles based on personal and/or identifying user information and/or user responses to profile setup questionnaires. A user sensitive to a specific trigger and/or set of unrelated triggers may benefit from a custom profile through which they can have content filtered to their personal triggers and preferred remedial actions to those triggers. The individual trigger selection processes 414 may be used to generate custom stress profiles by allowing users to select specific triggers from lists, which may occur instead of or in combination with the processes 412-413. The individual trigger selection processes 414 may include methods which may provide users with an ability to select one or more triggers from a list (e.g., the trigger databases 417). The user profile update processes 415 may update stress profiles based on user feedback and/or data retrieved from input devices 303 (e.g., a profile may be updated to include new triggers and/or trigger severity based on heart rate data retrieved via a smart watch may indicate a user's stress response to triggering events). The user profile update processes 415 include methods for prompting users for information relating to their profiles, triggers, trigger severity, filtering accuracy, and/or other topics.

The system 400 may include the databases 417-419, which may be combined and/or further divided. The trigger databases 417 may include data such as lists of common triggers and/or lists of various indicators corresponding to triggers (e.g., visual indicators, audio indicators, text indicators, metadata indicators, closed captioning indicators, etc.). The trigger databases 417 may be updated (e.g., with new triggers, new indicators for existing triggers, etc.) via processes which may search external sources (e.g., internet encyclopedias, medical journal articles, social media platforms, etc.) for information about triggers and/or via feedback from users.

The general stress profile databases 418 may include pre-generated general stress profiles (e.g., general profiles created via the general stress profile generation processes 412). The custom stress profile databases 419 may comprise stress profiles generated for particular users and associated data, including filtering preferences, personal and/or identifying information, questionnaire answers, and/or designated triggers.

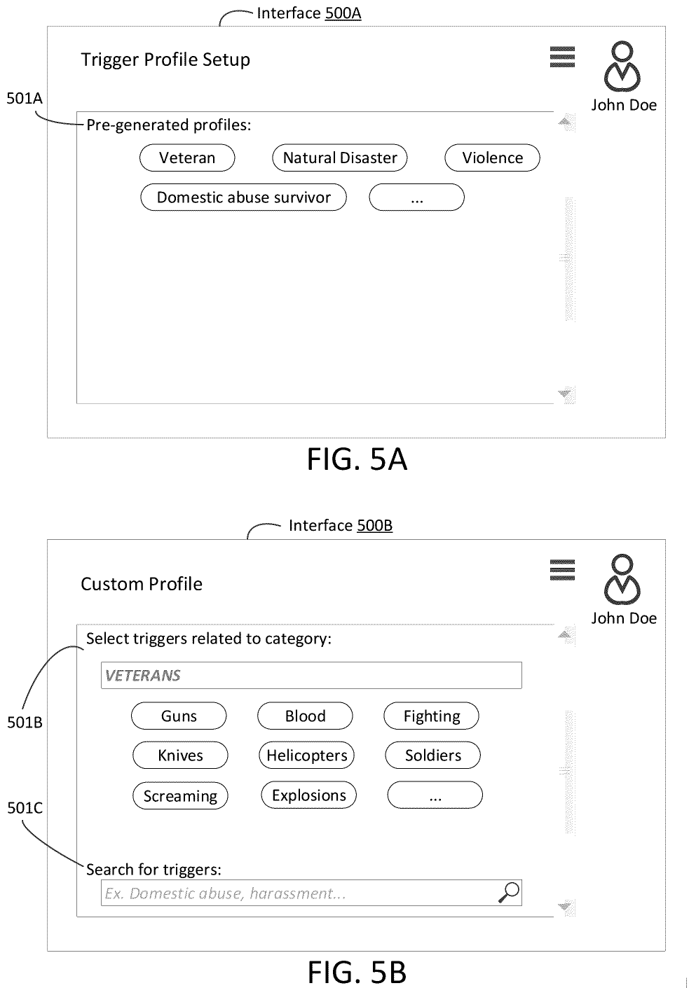

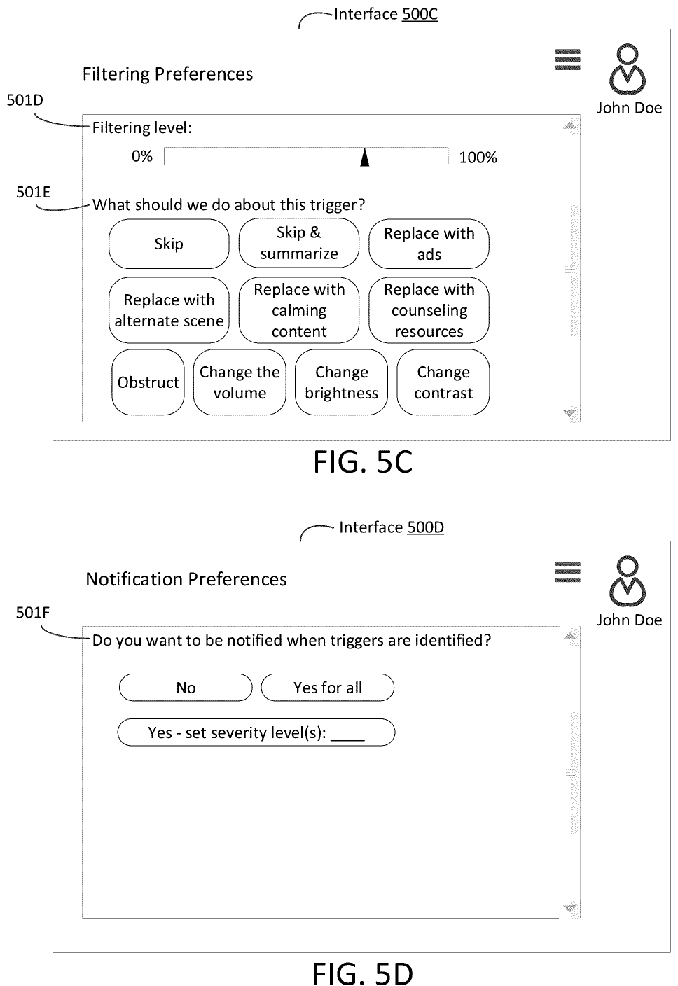

FIGS. 5A, 5B, 5C, and 5D show example interfaces 500A, 500B, 500C, and 500D for user profile setup. The interfaces 500A-D may be implemented by any device(s) and/or process(es) described above, and their use will be discussed in further detail below in FIGS. 17A-B. Filtering and/or applying remedial actions to content may depend on user information gathered via the interfaces 500A-D. The stress profile setup interfaces 500A-B may allow a user to begin selection and/or creation of profiles for identifying stress triggers using a questionnaire, which may display pre-generated stress profiles 501A to users, allow users to select triggers based on categories 501B, and/or allow users to search for specific triggers via the general search 501C. The pre-generated profiles 501A may be generated based on common categories of users (e.g., veterans, domestic abuse survivors, etc.) who may experience stress reactions, PTSD, etc. due to certain types of triggers. A user who is a combat veteran may for example, not want to select the pre-generated “Veteran” profile if not all of the included triggers apply to the user, so the user may choose to search for triggers related to the category 501B veteran, and choose only the triggers relevant in their custom profile. For a user who is sensitive to potentially unrelated triggers (e.g., tornados and guns), the general search 501C may allow the user to search all of the known triggers (e.g., stored in the trigger databases 417) for specific triggers, and generate a custom profile for filtering the selected triggers.

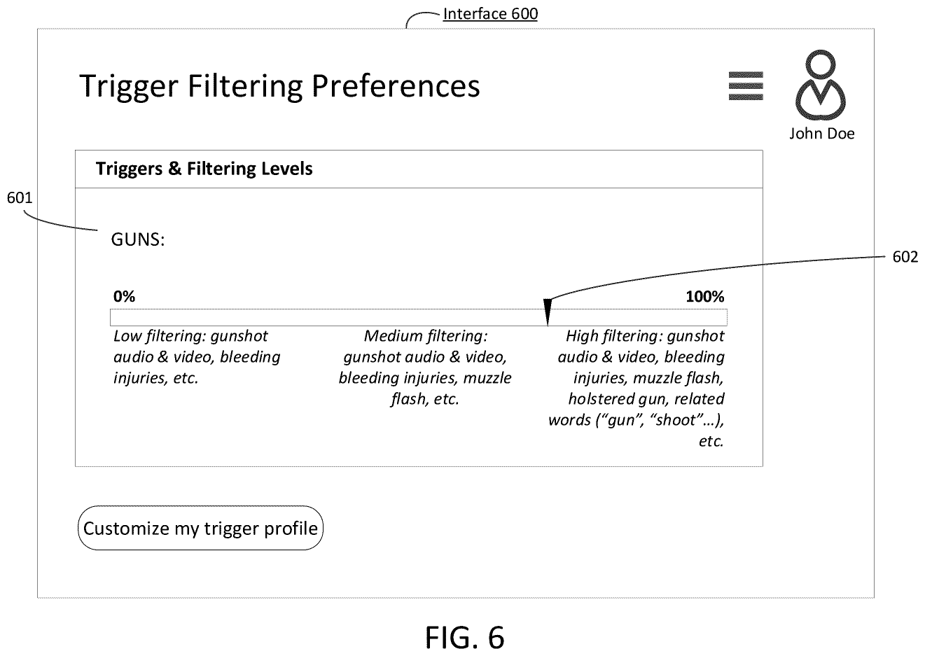

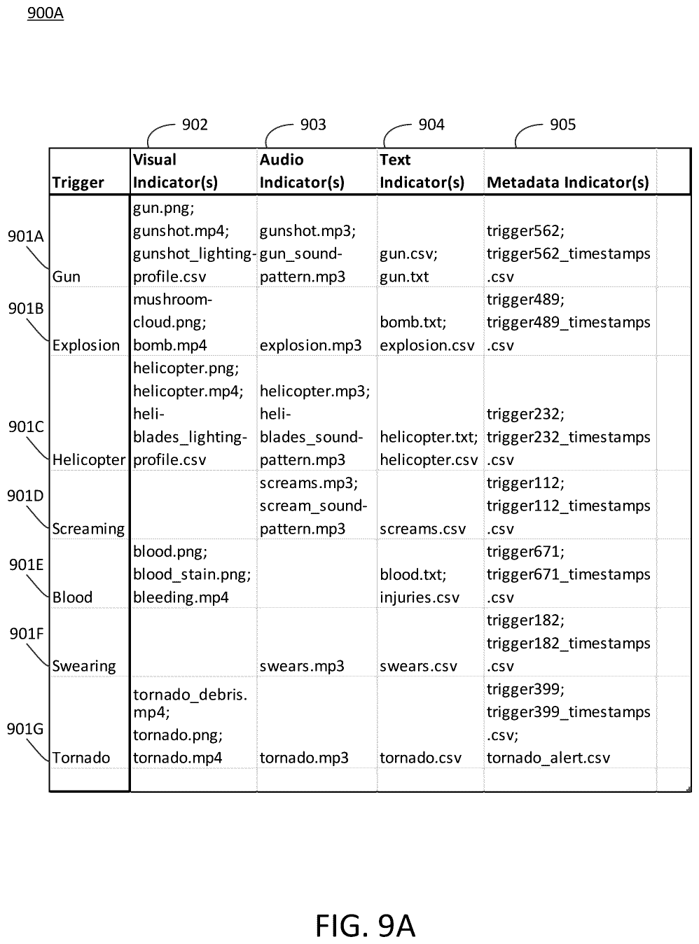

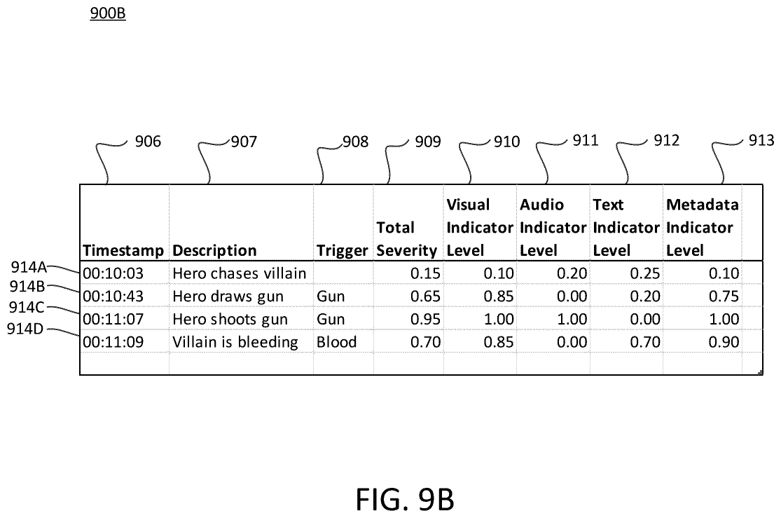

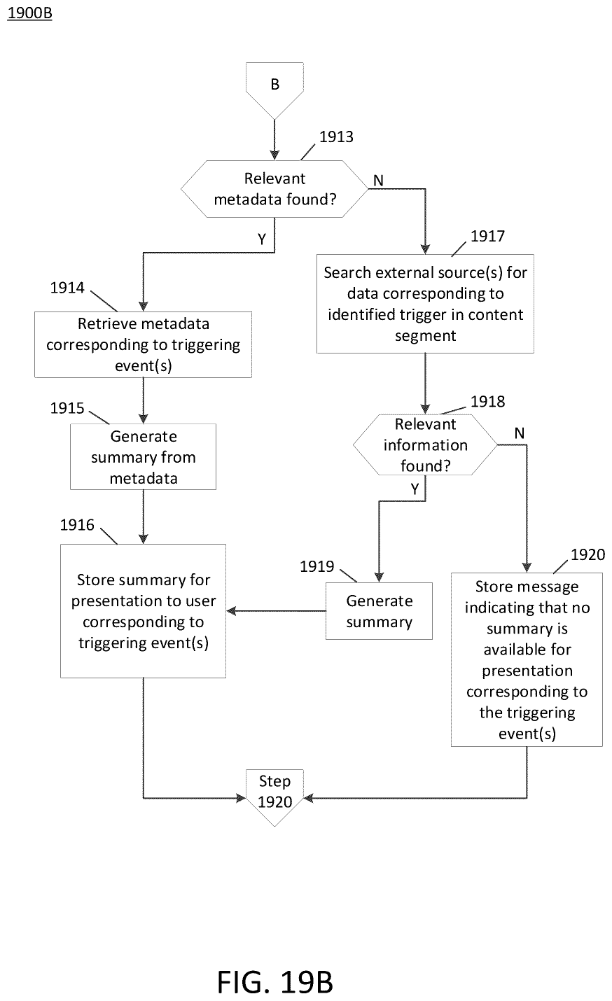

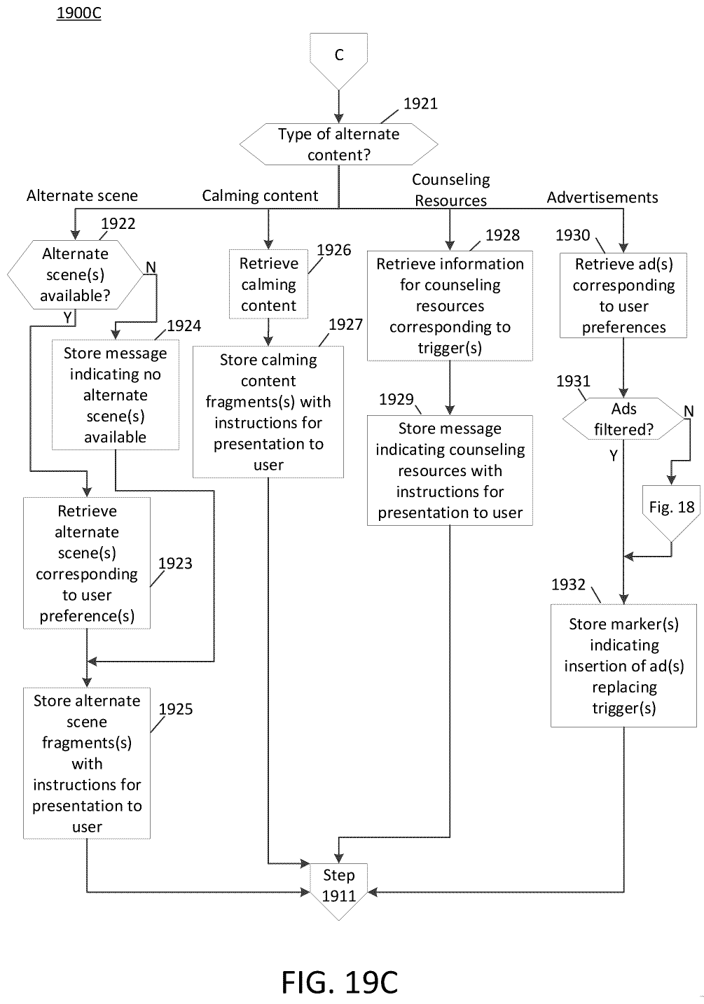

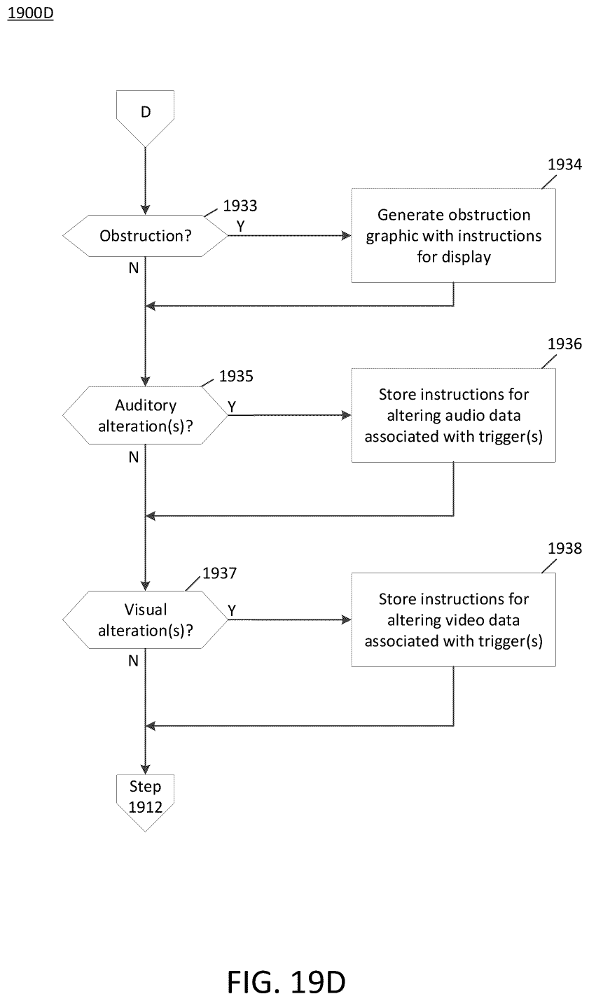

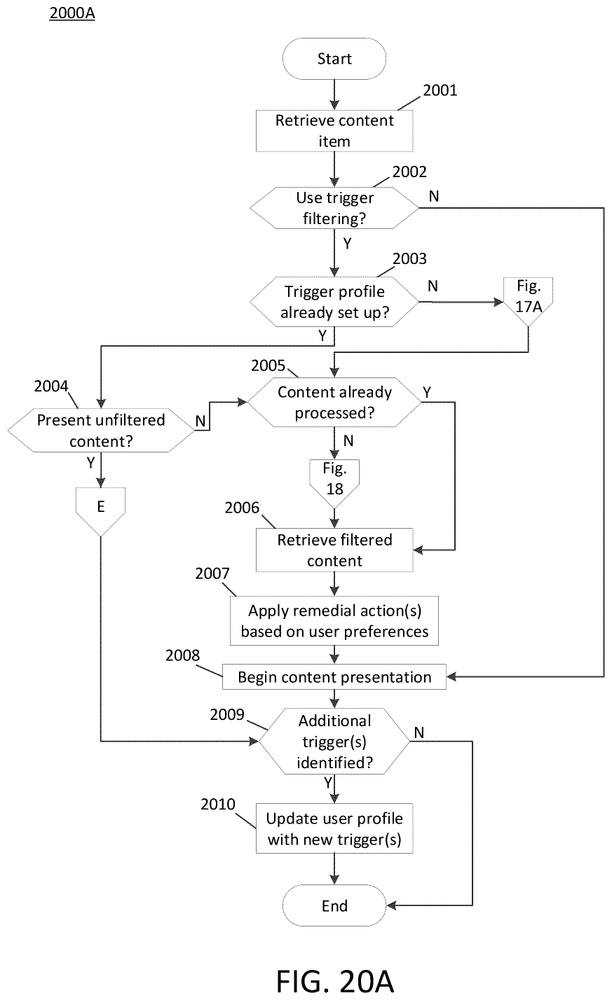

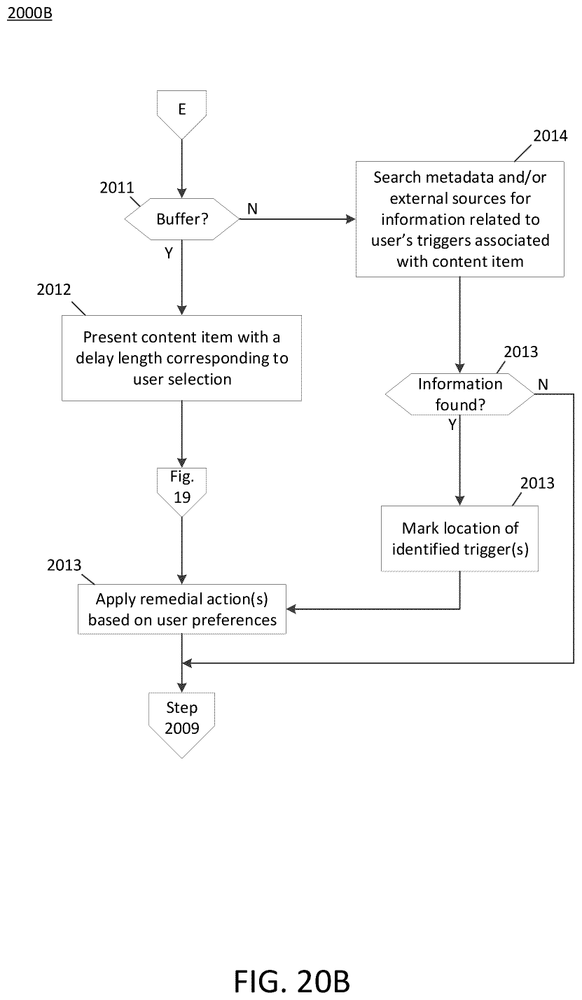

The interface 500C may allow users to select preferences for the stress filtering, and may include a filtering level selector 501D which may indicate the degree to which a user would like certain triggers may be filtered, where a higher percentage may indicate more sensitive filtering and a lower percentage may indicate more lax filtering, which will be further detailed below for FIG. 6. Option 501E may display options for different remedial actions which may be implemented based on identification of triggers indicated by the user, including skipping triggering events, skipping and displaying a textual summary of the triggering events, replacing the triggering events with various types of alternate content (e.g., with advertisements, alternate scenes, calming content, counseling resources, etc.), and/or altering the triggering events (e.g., via obstructing a portion of the content showing triggers, changing the volume, brightness, and/or contrast associated with the triggering events). The remedial actions are further detailed below for FIGS. 20A-D. The notification preference interface 500D may allow users to select notification preferences associated with content filtering. Options 501F may allow a user to indicate whether they would like to be notified of all identified triggers, triggers meeting certain severity levels, or not at all. For example, a user sensitive to gun violence may prefer to receive notifications every time a gun is identified, or the user may prefer to only receive for very guns with high severity levels (e.g., a gunshot occurs onscreen). Trigger severity levels will be further discussed below for FIGS. 9B and 19.

FIG. 6 shows an example stress profile interface in which filtering levels may be determined, including an interface 600, which illustrates a selected trigger 601 and its associated filtering preferences 602, which may be implemented by any device(s) described above to allow users to specify a level of filtering that they wish to apply. The selected trigger 601 may be guns, and the associated filtering preferences 602, for example, may be set to a moderate level (e.g., a user may select a filtering level of approximately 70%, which may indicate that the user wants guns identified in content to be remediated if their identified indicators include those with high severity, such as clear images/sounds of gunshots, clear images of bleeding/gunshot wounds, as well as those with moderate severity, such as images/lighting profiles of a muzzle flash, a holstered gun, etc.). Further discussion of trigger severities and filtering levels follows below relating to FIG. 9B. The filtering preferences 602 may be denoted by a slider where a low filtering level may indicate that filtering should occur only when high-severity indicators of the trigger are identified (e.g., gunshot audio, gunshot video, bleeding gunshot injuries, etc.), a moderate filtering level may indicate that filtering should occur when moderate-severity indicators are also identified (e.g., a gunshot muzzle flash), and/or a high filtering level may indicate that filtering should occur when low-severity indicators are also identified (e.g., a holstered gun, dialogue including related words such as “gun”, “shoot”, etc.). A user viewing an action film filtered according to this profile may watch scenes including gunshots filtered and/or remediated at a moderate-to-high level, which may mitigate symptoms of PTSD associated with viewing scenes containing severe and moderate indicators of guns.

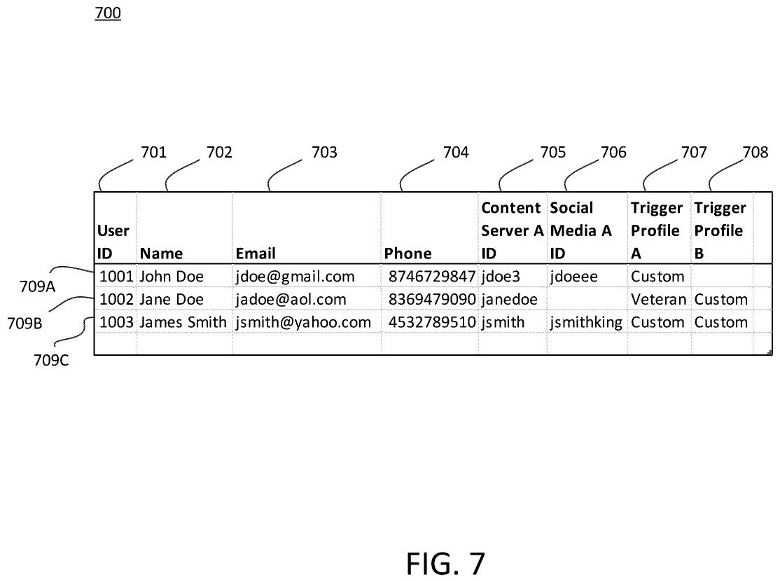

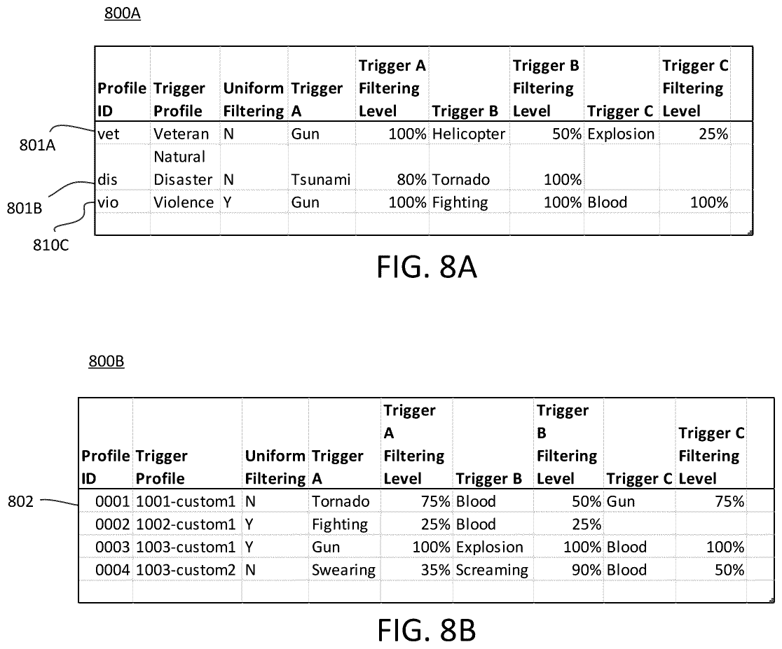

FIG. 7 illustrates an example database 700 of user accounts, which may include a variety of user information including personal and/or identifying information (e.g., that may be linked to a user's filtering preferences. Presentation of content to users (described below corresponding to FIG. 21) may include steps to check the database 700 for information indicating whether users have configured stress profiles. The database 700 may be implemented by any device(s) described above. A user account 709C may, for example, indicate a user James Smith's social media ID 706 and stress profiles 707-708. The user's social media activity may be examined to learn about their triggers (e.g., James may have created a social media post discussing a triggering scene in a movie, which may be used to indicate a type of trigger that should be filtered). The account 709C may indicate the stress profiles generated for the user. James may have two custom profiles linked to his account for case of reference, which may be configured to filter different categories of triggers organized based on James' personal preferences (e.g., one custom profile may be configured to filter triggers relating to car crashes, the other custom profile may be configured to filter blood and screaming, etc.). The account 709B for example user Jane Doe may include a pre-generated stress profile for veterans and a custom stress profile (e.g., she may select the Veteran profile and also generate a custom profile based on other unrelated triggers). Further discussion of general and custom stress profiles follows below, corresponding to FIGS. 8A-8B.

Groups of stress triggers may be stored as profiles for ease of reference. FIGS. 8A and 8B illustrate example stress profile databases 800A-B, which may store information indicating groups of stress triggers organized by profile. Description of user profile setup processes in FIG. 17 below includes details relating to the generation of stress profiles stored in the databases 800A-B. For example, a combat veteran profile 801A may indicate types of stress triggers commonly associated with veterans who suffer from PTSD. The combat veteran profile 801A may indicate that the user may experience stress when they see (and/or hear) a gun (Trigger A), helicopter (Trigger B), or explosion (Trigger C). The profiles may be predetermined 800A (e.g., profiles for expected types of susceptibility, such as combat veteran 801A, disaster survivor 801B, domestic violence survivor 801C, etc.), and/or customized 800B according to a particular user's susceptibility. Each trigger in the profiles may have an associated filtering level, indicating the degree to which that particular trigger should be handled (as mentioned above relating to FIG. 6 and described below). The account 709B associated with the user Jane Doe may implement the combat veteran profile 801A, and content she views may be automatically filtered based on profile 801A may alleviate Jane's symptoms associated with viewing content including, for example, scenes of war showing gunfights, explosions, and helicopters. The custom stress profile database 800B may comprise the same features as the database 800A, associated with profiles based on specific users' susceptibilities. Custom stress profiles may include related and/or unrelated triggers, which may be designate by users during configuration processes (e.g., set up processes) detailed below. A user may be sensitive to unrelated triggers which may include tornados and blood, for example, illustrated by the custom stress profile 802, which shows different filtering levels for each trigger based on the user's preferences. The profile 802 may consolidate the user's unrelated triggers and corresponding preferences for convenience during the profile setup process.

FIG. 9A illustrates an example trigger database 900A indicating characteristics of different types of triggers, which may be used during content analysis for trigger identification. The database 900A may be implemented by any device(s) described above. The database 900A may include Trigger Name 901, Visual Indicator(s) 902, Audio Indicator(s) 903, Text Indicator(s) 904, and/or Metadata Indicator(s) 905. The visual indicators 902 may include image files, video files, text files, and/or other types of files which may define visual components associated with a trigger. The audio indicators 903 may include audio patterns of a trigger, sound pattern files, and/or other types of files which may define audio associated with a trigger. The text indicators 904 may include text files (e.g., transcripts, closed captioning data, content summaries, etc.) which may define text associated with a trigger. The metadata indicators 905 may include trigger codes (e.g., “trigger562” for gun, etc.), timestamps of trigger locations within content items, manifest files associated with content items, and/or other types of files. Trigger entries 901A-901G may comprise data associated with the categories 901-905. The entry 901A “Gun” may, for example, include images, videos, lighting profiles, sounds, sound patterns, words and/or phrases related to guns, gun trigger codes, and/or timestamps for gun occurrences in an example content item. Identification of a gun in a content item may depend on finding any one or more of the indicators associated with the entry 901A. The filtering process may then look for these audio patterns, video patterns, textual patterns, and/or metadata indicators to determine the presence of a stress triggering event.

FIG. 9B illustrates example data table 900B indicating characteristics of stress trigger events that have been detected in a filtered content segment. For example, a movie may include a scene in which a hero chases and shoots a villain using a gun. The movie may be processed using the profiles 800A/B and trigger characteristics 900A to identify stress trigger events in the movie. In the illustrated example, a gun in entry 914B is detected at the 10:43 time point in the movie, in which the hero draws their gun. This stress trigger event may be detected by performing computerized image recognition to match an image pattern of a gun. Since the hero has only just drawn their gun, and has not fired it yet, the severity 909 may be relatively low as the gun is only recognized by a partial image with no other indicators of gun violence. The severity 909 may be based on how clearly the stress trigger was identified (e.g., a 1.00-level visual indicator may denote a full view of a gun; a 0.20-level audio indicator may denote a minor sound associated with guns, such as releasing of a safety; etc.). The trigger severity 909 may be based on a variety of factors including some combination of the indicator levels 910-913 (e.g., an average value and/or other calculation based on the indicator levels 910-913 may indicate the severity 909 of a trigger in a segment). The table 900B may include entries 914A-914D corresponding with filtered segments of the scene, comprising data associated with the categories 906-913.

In the example scene where the hero chases and eventually shoots the villain, the entry 914A may indicate a stress trigger that is based only on textual dialog suggesting gun violence (e.g., a low 0.25-level dialogue based on recognizing the phrase, “I don't want to shoot!” in a closed-captioned feed of the movie), resulting in a low 0.15-level total trigger severity. The low severity may be due in part to the fact that no gun was seen or heard. The entry 914B may indicate a higher severity stress trigger as the gun is now visible when the hero corners the villain and draws a gun (e.g., indicating a higher 0.85-level visual indicator). The entry 914C may indicate a stress trigger based on the hero actually shooting the villain, with a high 1.00-level visual indicator (e.g., a clear image of the gun is recognized in the movie frame), a high 1.00-level audio indicator (e.g., a loud on-screen gunshot is detected in the movie audio), resulting in a high 0.95-level total trigger severity. The entry 914D may indicate a stress trigger event in which the villain is left bleeding on the ground, including a high 0.85-level visual indicator (e.g., image recognition detected image of blood in the movie frame). For the same content segments, trigger filtering levels associated with various trigger filtering profiles may yield different filtering results. For example, the combat veteran profile 801A indicates a gun trigger filtering level of 100%, and the custom stress profile 802 indicates a gun filtering level of 75%. If the example scene is filtered based on the combat veteran profile 801A, the 100% filtering level may trigger remedial actions (e.g., a message indicating links to veteran support organizations) for guns identified with any severity level. If the example scene is filtered based on the custom profile 802, the 75% filtering level may trigger remedial actions for guns identified with severity levels greater than 0.75, so the gun identified in 914C may trigger a remedial action. Remedial actions may be based on the filtering level indicated in the stress profile, for example, a 100% filtering level for guns may be associated with remedial actions offering counseling resources, and a 75% filtering level for guns may be associated with a remedial action skipping the scene containing the trigger.

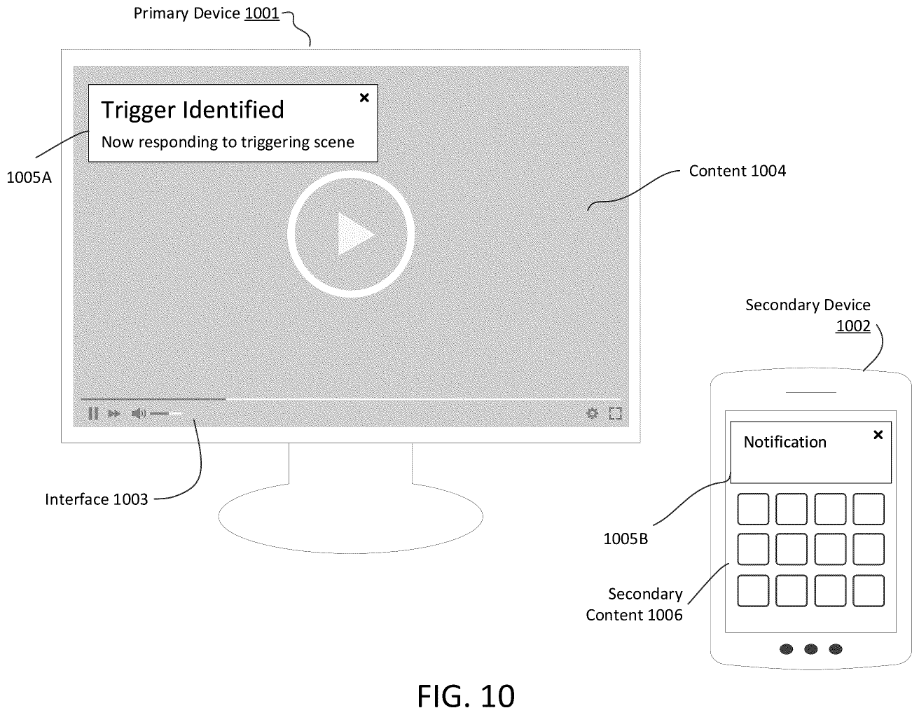

FIG. 10 illustrates an example system of devices which may implement the features described above, including primary devices 1001 that is used to output a content item (e.g., displays including televisions, personal computers, laptop computers, etc.) and/or secondary devices 1002 that may serve as a second-screen for the viewing of the content item (e.g., smartphones, tablet devices, laptop computers, personal computers, mobile devices, gaming consoles, wireless devices, etc.). The primary device 1001 may comprise an interface 1003, may display content 1004, and/or may display notifications 1005A. The secondary device 1002 may display secondary content 1006 and/or notifications 1005B. The notifications 1005A-B may relate to trigger identification and/or correction. The secondary content 1006 may comprise webpage(s), social media platforms, online retailers, etc. A user may be watching a movie via a primary device (e.g., a television) and a triggering event may be identified in the movie (e.g., in the content 1004). The user may be using a secondary device (e.g., a smartphone) to browse social media (e.g., the secondary content 1006). Notifications 1005A-B relating to the identified trigger and/or remedial actions may be displayed on one or more of the devices 1001-1002, alerting the user to the identified trigger they may have missed while browsing social media, which may mitigate PTSD symptoms since the user was not surprised by the trigger.

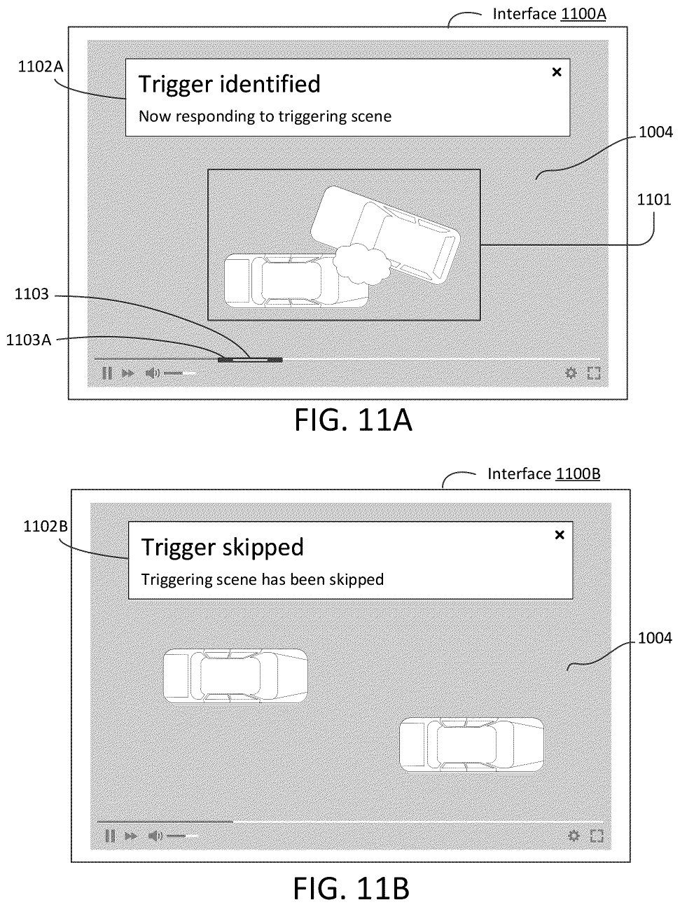

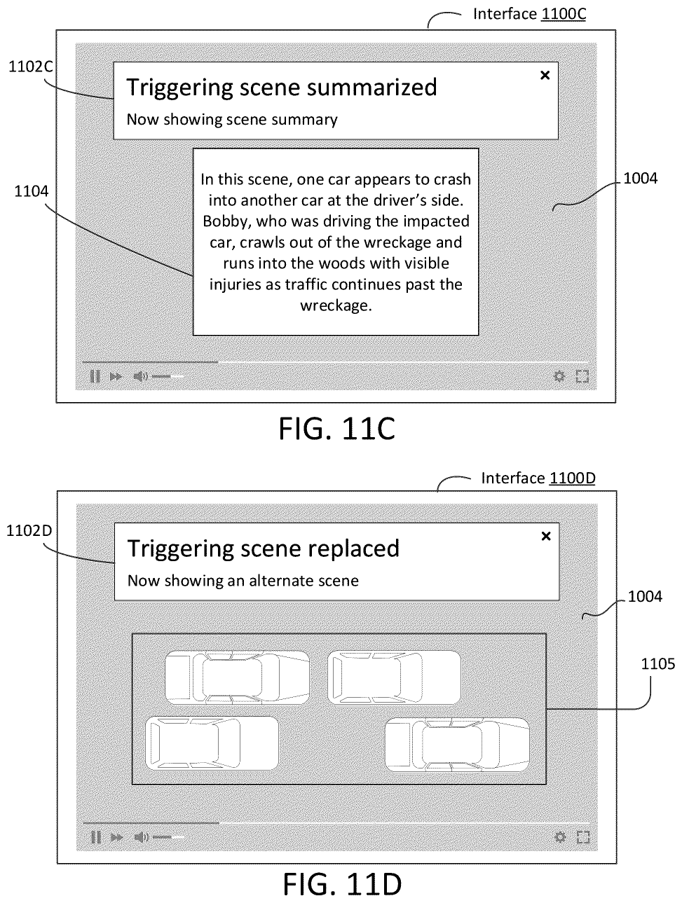

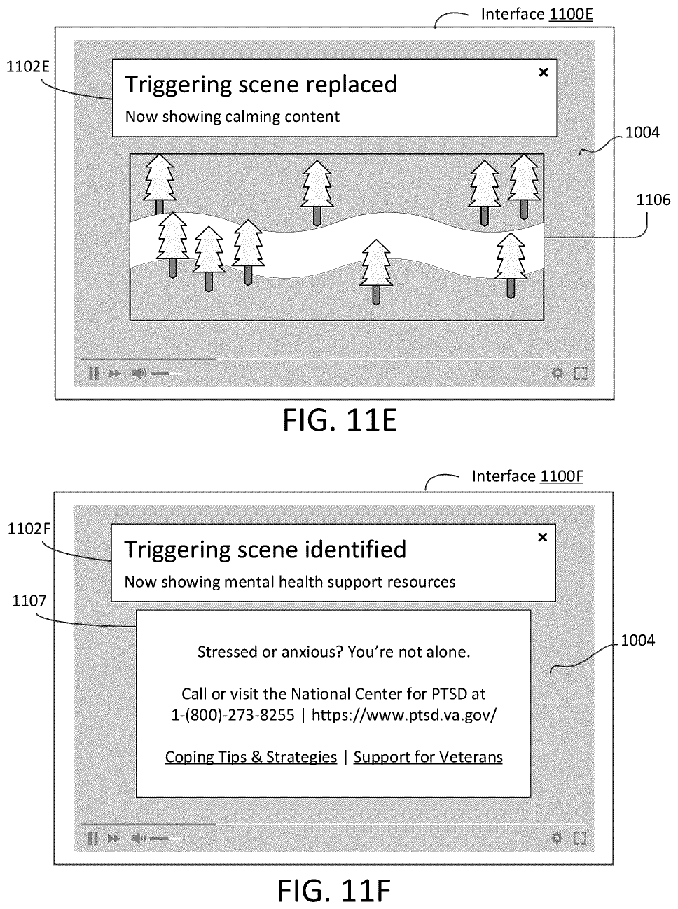

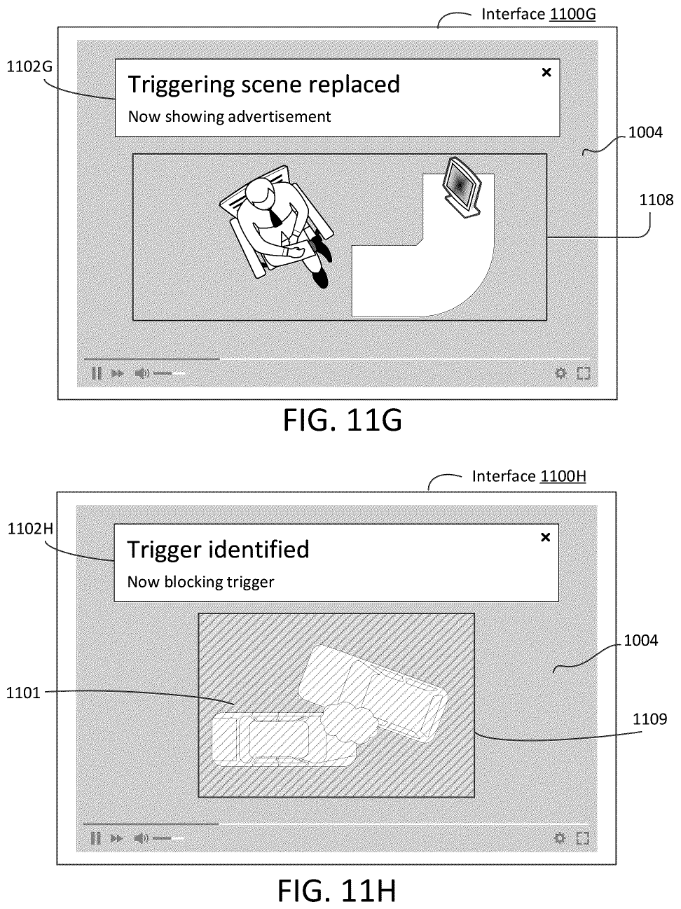

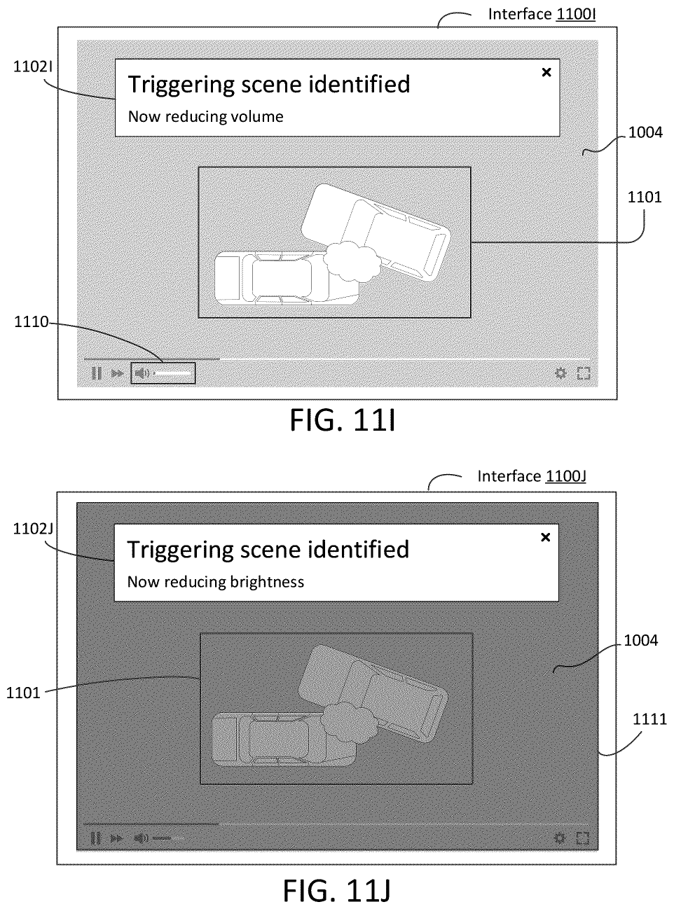

FIGS. 11A-H show example interfaces 1100A-H illustrating identification of a triggering event (e.g., a car crash) in a content item, and/or remedial and/or remedial actions. The interfaces 11A-H may be implemented by any device(s) and/or process(es) described above. Details associated with the implementation of the remedial processes are further discussed below relating to FIGS. 20A-D. FIG. 11A is an example interface 1100A in which a car crash has been identified as a possible stress trigger for the user viewing the content item. The interface 1100A may include the content 1004 (e.g., a movie), a triggering event 1101 (e.g., portion of the movie video frame showing a car crash), a notification 1102A that the triggering event was detected, triggering event boundaries 1103 indicating a segment of time, in the content, associated with the triggering event, and/or extended triggering event boundaries 1103A. The boundaries 1103, 1103A may be indicated by SCTE-35 markers and/or other SCTE-35-type markers; these markers may be comprised in manifest files associated with the content 1004. The triggering event 1101 (e.g., a car crash) may be identified in the content 1004. The notification 1102A notifying a user of the identified trigger may be displayed. The triggering event boundaries 1103 may denote the start and/or end times of the triggering event in the content (e.g., movie); the extended boundaries 1103A may be based on lead-up and/or comedown events related to the identified trigger which may also be triggering to a user (e.g., screeching brakes may be a lead-up event that may trigger a user sensitive to car crashes). Use of the extended boundaries 1103A in trigger identification may allow even earlier advance warning of a triggering event, and/or even later offers of remedial actions.

FIG. 11B is an example interface 1100B showing an example trigger skipping remedial action. The interface 1100B may comprise the content 1004 and/or a notification 1102B (e.g., a triggering event response notification and/or a trigger skipping notification). When a triggering event is skipped, a user may continue viewing the content 1004 and/or the notification 1102B may alert the user to the skipping remedial action performed. FIG. 11C is an example interface 1100C, showing an example content summarizing remedial action. The interface 1100C may comprise a notification 1102C (e.g., a triggering event summary notification), the content 1004, and/or triggering event summaries 1104. The summary 1104 corresponding to the identified triggering event 1101 may be displayed over the segment comprising the trigger and/or it may be combined with the skipping action in FIG. 11B. A summary of triggering events may be displayed during playback of the triggering events and/or after the triggering events are skipped, allowing users to learn the details of the content segment without being subjected to events causing their stress triggers.

FIGS. 11D, 11E, 11F. 11G are example interfaces showing example content replacing remedial actions. FIG. 11D shows an example content replacing response, wherein a triggering event may be replaced by an alternate scene 1105. An interface 1100D may comprise a notification 1102D (e.g., a triggering event replacement notification), the content 1004, and/or the alternate scene 1104A. Alternate scenes may comprise scenes which may be rated for general audiences, and/or scenes determined to be suitable for certain users (e.g., machine learning models may be able to predict suitable alternate scenes for users based on known user information), and/or other content. FIG. 11E illustrates an example content replacing response, wherein a triggering event may be replaced by a calming scene 1106. An interface 1100E may comprise a notification 1102E (e.g., a triggering event replacement notification), the content 1004, and/or the calming scene 1106. Calming scenes may comprise nature scenes and/or segments of from a certain movie comforting (e.g., scenes from a childhood favorite movie), which may be indicated in a profile. FIG. 11F shows an example content replacing remedial action, wherein a triggering event may be replaced by a message 1107 which may indicate counseling resources. An interface 1100F may comprise a notification 1102F (e.g., a triggering event replacement notification), the content 1004, and/or the message 1107. The message 1107 may include contact information for PTSD support organizations, guided meditations, links to websites indicating coping strategies, etc. FIG. 11G shows an example content replacing remedial action, wherein the triggering event may be replaced by advertisements 1108. An interface 1100G may comprise a notification 1102G (e.g., a triggering event replacement notification), the content 1004, and/or the advertisements 1108. The car crash scene may be replaced by an advertisement (which may be filtered according to the user's preferences), providing a distraction from the triggering events, which may aid in alleviating PTSD symptoms associated with the car crash scene.

FIGS. 11H, 11I, 11J include example interfaces showing example content altering remedial actions, in which the content item may be altered to deal with a triggering event. FIG. 11H shows an example content altering remedial action, wherein the scene comprising triggering events may be altered via blocking at least a portion of the triggering events. An interface 1100H may comprise a notification 1102H (e.g., a trigger altering notification), the content 1004, the triggering event 1101 and/or a triggering event obscuring graphic 1109. The graphic 1109 may comprise shading and/or blocking at least an area of the content 1004 which may correspond to the triggering event 1101. FIG. 11I shows an example content altering remedial action, wherein the scene comprising triggering events may be altered via changing the associated audio. An interface 11001 may comprise a notification 1102I (e.g., a trigger altering notification), the content 1004, the triggering event 1101, and/or a volume indicator 1110. The volume indicator 1110 may represent various changes to the associated audio, including decreased and/or silenced audio. A user triggered by sounds of car crashes may continue consuming a scene comprising a car crash with lowered volume, which may not induce their symptoms. FIG. 11J shows an example content altering remedial action, wherein the visual components of the scene containing triggering events. An interface 1100J may comprise a notification 1102J (e.g., a trigger altering notification), the content 1004, the triggering event 1101 and/or a triggering event visual alteration 1111. Altering video associated with triggering events may comprise altering brightness and/or contrast corresponding to at least an area of the content 1004 which may correspond to the triggering event 1101. A user triggered by bright flashes resulting from an exploding car crash scene may continue watching the scene with reduced brightness, which may allow them to consume the triggering event, which may not induce their PTSD symptoms.

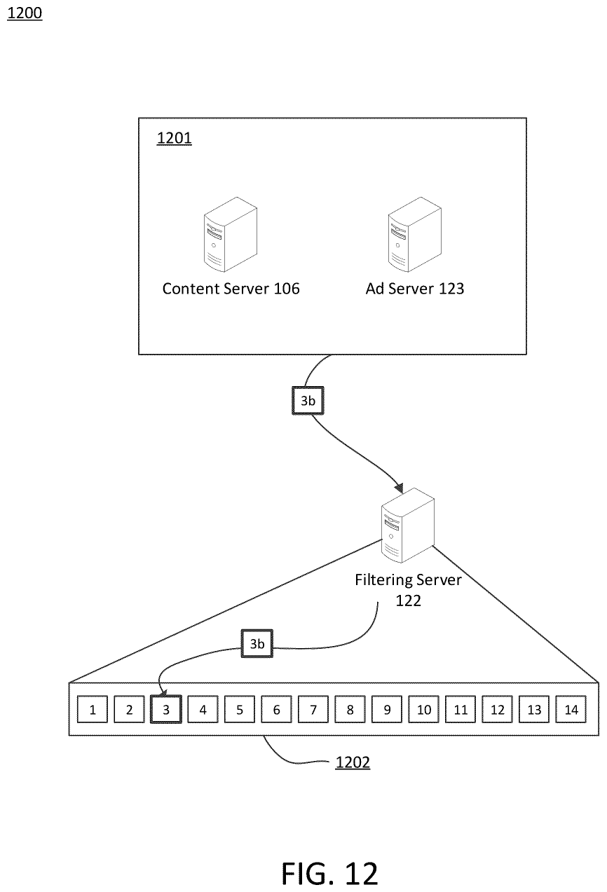

FIG. 12 shows an example system 1200 via which manifest files 1202 may be altered. An environment 1201 may comprise, for example, the content server 106, the advertisement server 123, other content servers, filtering servers, and/or any other device(s) and/or process(es) described above. The environment 1201 may comprise the filtering server 122. The manifest files 1202 may be retrieved by the filtering server 122 via content servers (e.g., the content server 106, the advertisement server 123, etc.). The manifest files 1202 may comprise data relating to content fragments, as denoted by the boxes labeled 1-14 and 3b in FIG. 12. For a user indicating a preference for replacing a trigger (e.g., a gunshot) comprised in fragment 3 with alternate content, machine learning models implementing trigger filtering may request the replacement with content fragment 3b. The manifest file 1202 may be amended with the fragment 3b using SCTE-35 markers and/or other standards. The filtered and/or remediated content may be sent to the user's device(s) for playback in the form of a new manifest file comprising fragments 1, 2, 3b, 4-14. Remedial actions (e.g., illustrated in FIGS. 20A-D) may include steps indicating updating of manifest file(s), as described above for replacing fragment 3 with fragment 3b, which may be implemented according to the system 1200.

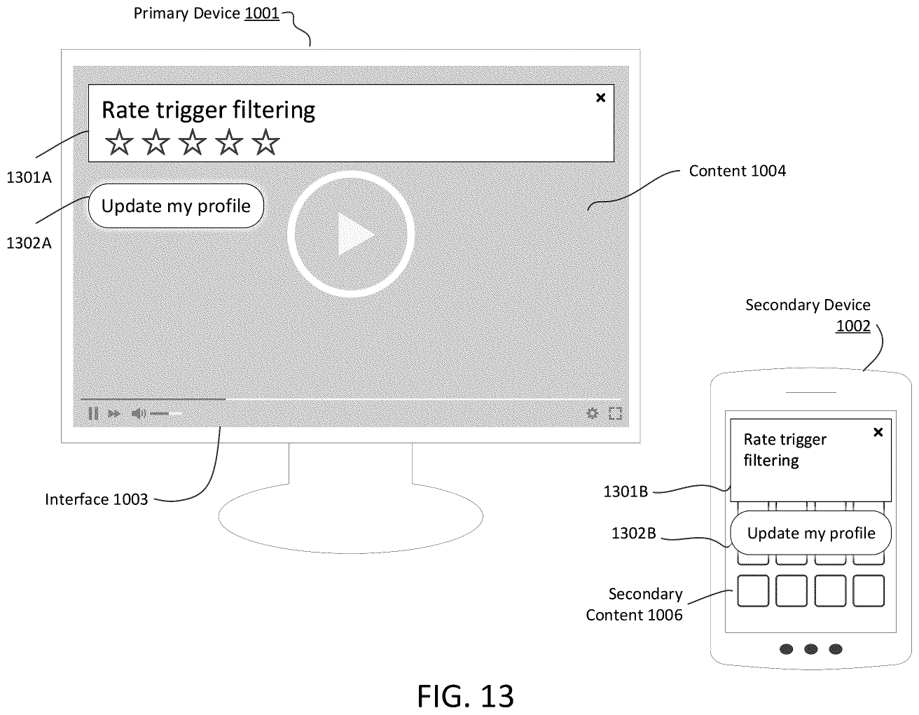

FIG. 13 illustrates example filtering feedback interfaces of secondary devices 1002 in association with primary devices 1001, which may be implemented by the examples in FIG. 10. The primary device 1001 may comprise the interface 1003, the content 1004, a feedback prompt 1301A, and/or a profile customization button 1302A. The secondary device 1002 may display the secondary content 1006, a feedback prompt 1301B, and/or a profile customization button 1302B. The feedback prompts 1301A-B may comprise ratings on a scale (e.g., star ratings) through which a user may provide information indicating qualitative and/or quantitative feedback relating to filtered content, user profiles, etc. A user may watch filtered content via devices 1001 and/or 1002. The user may receive the prompts 1301A-B for feedback regarding the filtering, which may be used to optimize machine learning models and/or further customize user data. The user may have the option update their profile and/or filtering preferences via the profile customization buttons 1302A-B, which may appear on the screen in combination and/or separately from the feedback prompts 1301A-B.

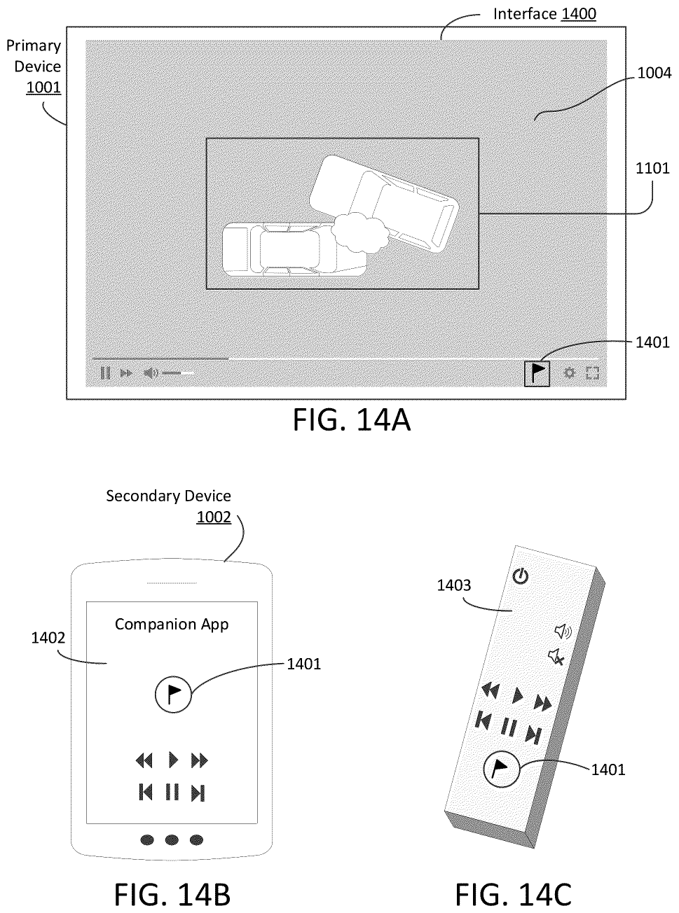

Sometimes, a triggering event is not recognized in advance, or is not part of the user's existing profile, and the user wishes to mark a scene as a triggering event for future reference and/or to improve accuracy of future trigger detection. FIGS. 14A, 14B, and 14C illustrate example trigger flagging options, in which a user may mark a portion of a content item as containing a triggering event. FIG. 14A comprises the primary device 1001, an interface 1400, the content 1004, the triggering event 1101, and/or a flag button 1401. A user watching the content 1004 via the interface 1400 may user the flag button 1401 to indicate the occurrence of a trigger (e.g., a car crash). FIG. 14B comprises the secondary device 1002, an interface 1402 and/or the flag button 1401. The interface 1402 may implement the same features as the interface 1400 via the secondary device 1002 comprising a filtering application (e.g., a virtual remote control, a companion viewing and/or filtering application, etc.). FIG. 14C comprises a device 1403 (e.g., a remote-control device), which may comprise the flag button 1401. In an example, a user may be watching content comprising a car crash on a television (e.g., the primary device 1001). The user may flag the triggering event occurrence using the flag button 1401 via the interface 1400, the interface 1402, the device 1403, and/or any combination thereof.

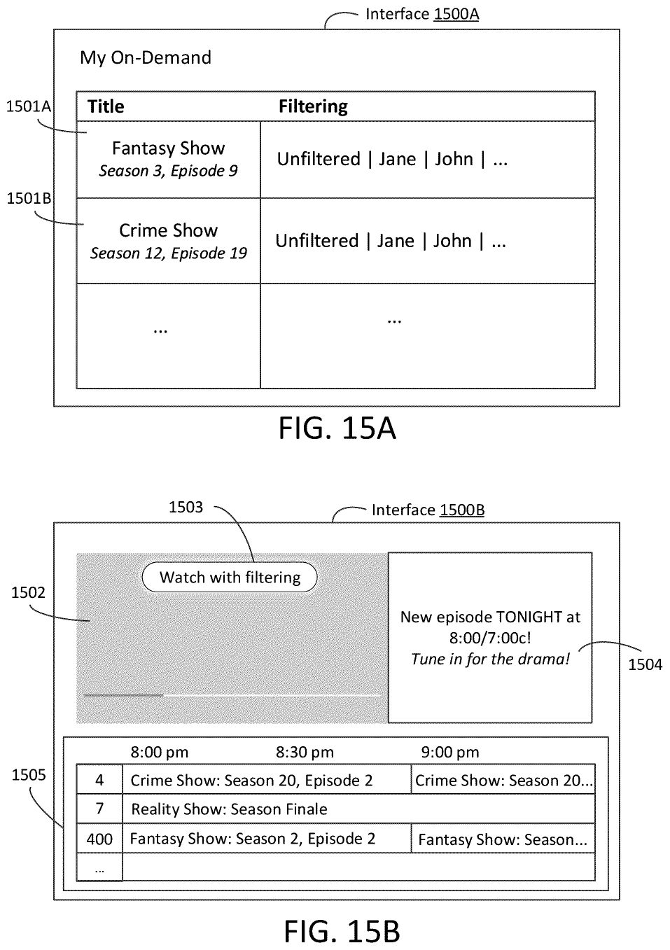

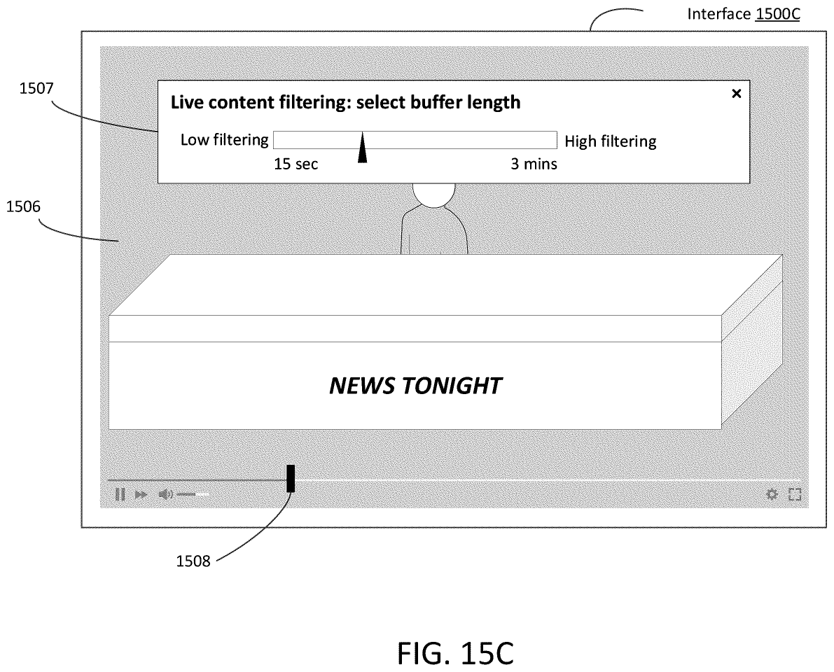

Content filtering may be applied to various types of content, including streaming and/or video-on-demand (VOD) content, multicast content (which may comprise broadcast content), and/or live content. FIGS. 15A-C illustrate example interfaces for filtering various types of content. FIG. 15A is an example interface for filtering streaming and/or VOD content. FIG. 15A may comprise an interface 1500A, and/or entries 1501A-B. The entries 1501A-B illustrate content, such as on-demand content, which may be viewed in unfiltered and/or filtered form based on preferences of one or more users. Example users Jane and John may have different filtering preferences, so they may view different versions of the same content item (e.g., the fantasy show in entry 1501A and the crime show in entry 1501B), filtered according to each of their respective preferences.

FIG. 15B is an example interface illustrating a program listing 1505 (e.g., indicating a schedule of times when various content items will be available for reception), showing an option that allows users to select a scheduled content item for viewing with stress trigger filtering enabled. FIG. 15B may comprise an interface 1500B, primary content 1502, a filtering button 1503, and/or secondary content 1504 (e.g., advertisements). The content corresponding to 1502 and/or 1504 may be filtered content (e.g., it may be preprocessed based on pre-generated and/or custom stress profiles associated with a user prior to being available for multicast). FIG. 15B illustrates the same features as FIG. 15A, but for scheduled multicast content instead of on-demand content. A user may access content items via the program listing 1505 and use the filtering button 1503 to select filtering viewing options. Within the interface 1500B, the user may view a small preview version of the selected primary content 1502 and the secondary content 1504. Selected filtering options may indicate that the secondary content 1504 is filtered, for example, advertisements for a movie containing the user's trigger(s) according to their profiles (e.g., as denoted in the database 700) may not be displayed, and instead replaced with other advertisements.

Computing devices may require some processing time in order to identify trigger events in content items, and this may present difficulties for live content items that are transmitted as they occur (e.g., live sporting events, live news broadcasts, etc.). To provide time for such processing, such live content items may be buffered, and their presentation delayed by an amount of time. The filtering system may use that time to process the audio/video/text/metadata of the content item, and to identify trigger events that may trouble viewers. The user may be given a choice as to whether they wish to view a filtered version of the live event, and if they wish to view a filtered version, the user may also be given a choice as to how much buffering time they are willing to wait for the live content. FIG. 15C is an example interface for filtering live content, comprising an interface 1500C, content 1506, a buffer prompt 1507, and/or a buffer delay indicator 1508. The live content shown in the listing 1505 may include news, weather reports, sports, etc.). The buffer prompt 1507 may prompt a user to select a buffer length, which may indicate the amount of time by which the live content 1506 may be delayed before presentation to the user. The buffer length may correspond to the thoroughness of filtering, where longer buffer times may allow for more detailed analyses and searches to identify current and/or upcoming triggers. The buffer delay indicator 1508 is a graphical symbol which may indicate the buffer length to the user. If the filtering is handled locally for the user (e.g., by a computing device that is providing the content item to the user), then the local filtering system may simply limit its filtering processes to what can be accomplished within the allotted time. If the filtering is handled remotely from the user, then the live content may be buffered for the user for the desired amount of time, resulting in a delayed presentation for that user, and the filtering will handle only those trigger events that were successfully identified in time to be of use to the user. Using this feature, if one user chooses a very long buffer length (e.g., 1 hour), and another user chooses a very short buffer length (e.g., 10 seconds), then each of the users may simply be presented with a content stream that is delayed by their respective buffer lengths, and their stress trigger features would simply be based on whatever triggers were successfully identified before the corresponding scenes were presented to the user. If a trigger event is detected after it has already been presented to the user, some remedial actions may still occur (e.g., offering users counseling resources, such as those illustrated in FIG. 11F). The event boundaries 1103/1103A may be used to determine whether such remedial actions should be taken (e.g., if a trigger is detected after the event has been showed to the user, remedial action may still be taken if the user's progress is currently within a time segment of the event boundaries 1103/1103A).

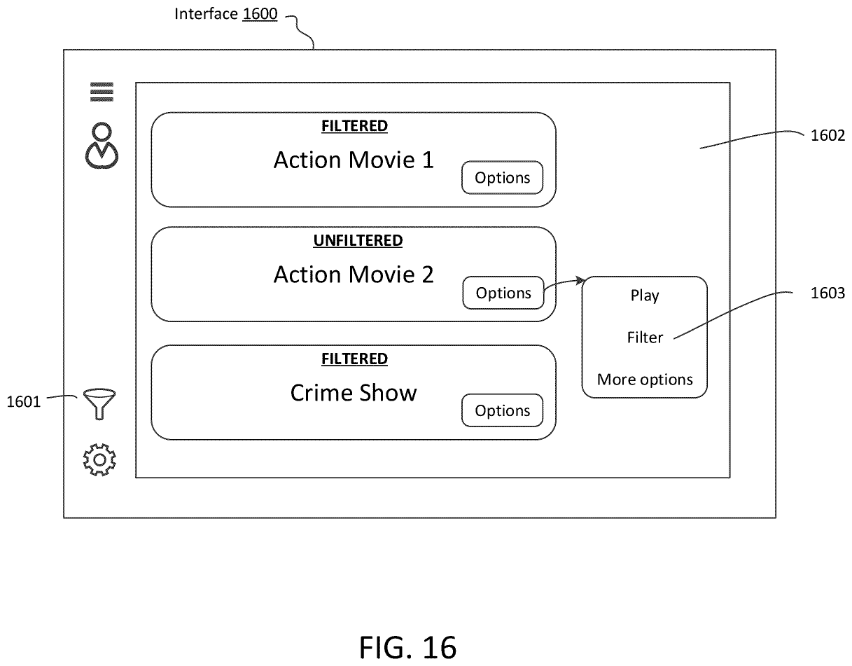

FIG. 16 is an example interface showing content items with indications of their filtered status (whether they have been processed to recognized stress triggering events), and with a sub-menu giving users the option to initiate filtering, view the content, etc. FIG. 16 may comprise a general interface 1600. The interface 1600 may comprise a filtering application button 1601, a filtering application interface 1602, and/or a filtering option 1603. The interface 1600 may be implemented by any device(s) described above. A display device (e.g., television, personal computer, etc.) may comprise a filtering application, which may be a native and/or stock application, denoted by the button 1601. The filtering application interface 1602 may comprise content items (e.g. movies, shows, etc.) which may be labeled as filtered if they were pre-processed (they may be labeled as unfiltered if they were not preprocessed). The filtering option 1603 may be comprised in an option menu associated with content items. By selecting the filtering option 1603, a user may indicate that a content item should be filtered. A user may implement the filtering application to filter content across any of the various content providers and/or servers described above, which may allow them to view content from any source in a filtered form to mitigate PTSD symptoms.

FIGS. 17A and 17B are flow charts showing an example process 1700A-1700B for user profile setup, to allow a user to configure the filtering system to identify trigger events to which the user is susceptible. Setting up a user profile may include displaying a questionnaire, retrieving demographic information, lists of selected and/or generated triggers, thresholds for filtering triggering content, and/or other user preferences. Searching for and/or identifying triggers in content items may be based on data comprised in user profiles. The profile setup interfaces in FIGS. 5A-D may implement the steps comprised in processes 1700A-B. The steps described in FIGS. 17A-B may be implemented by any of the various devices, processes, and/or machine learning models described above, including the user profile setup processes 403, the account database(s) 416, the trigger database(s) 417, the general stress profile database(s) 418, and/or the custom stress profile database(s) 419. The steps described herein may be combined and/or further divided.

In step 1701, a user may request to configure a stress profile (e.g., by selecting an option on a program listing displayed via a computing device), and options indicating types of stress profiles may be displayed. A user who is a tornado survivor may choose an option to configure their profile to help manage their PTSD with regard to tornados. The user may select and/or generate multiple stress profiles. The user may be presented with options for selecting from pre-generated profiles (e.g., a Natural Disaster profile in FIG. 5A), generating custom profiles, and/or selecting individual triggers. In step 1702, a user may be presented with options for configuring custom or pre-generated profiles. This may comprise the interfaces 500A-B, and may list a variety of pre-generated profiles 501A with triggers such as those shown in FIG. 8A, as well as options 501B-C for generating a customized profile. Note that while these are illustrated as alternatives, a user may employ both—e.g., selecting a pre-generated profile, and then customizing it, or adding a pre-generated profile to an existing customized profile, etc. In the event of overlapping triggers, the system may opt for the more sensitive filtering preference (e.g., the higher filtering level). If the user elects to configure a pre-generated profile, then step 1703 shows the list of pre-generated profiles 501A, and if the user selects one, such as the “Natural Disaster” profile in FIG. 5A, then the user's profile may be updated to reflect the selected pre-generated profile to automatically populate their profile with the triggers shown in the Natural Disaster profile 801B of FIG. 8A.

The questionnaire may be as shown in FIG. 5B, and may request that the user select one or more types of triggers that are vulnerabilities of the user. If the user elects to configure a custom profile, then in step 1704 the options for searching for triggers may be presented. For ease of display, the questionnaire may list triggers according to selectable categories. such as the pre-generated profiles discussed above. If the user wishes to see triggers corresponding to a specific category (e.g., if the user is a veteran but does not want to use the pre-generated “Veteran” profile, they may want to select individual triggers related to the category “veterans”), in step 1705, triggers correlated to the selected category may be displayed. While setting up a custom profile, a user may simply wish to select triggers based on a general search in step 1706. The user may be susceptible to seemingly unrelated triggers (e.g., tornados, guns, and blood), as shown in the custom profile 802 of FIG. 8B, so the triggers may be more easily selected via the general search 501C of triggers known to the system. The user's profile may then be updated to reflect the selected triggers.

In step 1707, the filtering level selector 501D may be displayed, as shown in FIG. 5C. The user may select a filtering level for each designated trigger. The filtering level selector 501D may be as shown in FIG. 6, describing examples of indicators that may be filtered at each filtering level. For the custom profile 802, the user may be prompted to select filtering levels for the triggers indicated, first for “Tornado” (e.g., where 75% may cause identification of a tornado at images of a tornado and a mere implication of a tornado via a character mentioning a tornado in dialogue), then for “Blood” (e.g., where 50% may cause identification of blood for graphic images of blood and graphic discussions of injuries by a character), and then finally for “Gun” (e.g., where 75% may cause identification of a gun for graphic visuals of gunshots, sounds of gunshots, lighting profiles indicating muzzle flashes, and certain graphic dialogue relating to guns), since the user may not be equally sensitive to all triggers within a profile. During profile setup, the user may be able to indicate that a certain filtering level may be applied uniformly across all triggers in a certain profile. The process 1700A may then proceed to step 1708.

After prompting the user to select filtering levels, step 1708 may display to the user the option to select remedial actions for their designated trigger(s). Step 1709 may display different types of remedial actions as shown in 501E in FIG. 5C. Remedial actions offered to the user may be as illustrated in the FIGS. 11B-J. They may include, for example, skipping the scene containing the trigger, skipping the scene containing the trigger and displaying a summary of events skipped, replacing the scene containing the trigger with advertisements, replacing the scene containing the trigger with an alternate scene that does not contain the trigger (e.g., a deleted scene that may not have made the final cut), replacing the scene containing the trigger with calming content (e.g., a gentle river), replacing the scene containing the trigger with a message indicating counseling resources (e.g., links to mental health support organizations), obstructing the trigger, changing the brightness, and/or changing the contrast. The user may change remedial actions which may have been included in pre-generated profiles.

In step 1710, the user may, for convenience, opt to have the system automatically select remedial actions based on trigger severity, input from the devices 303 (e.g., heart rate data indicating the user's stress level due to certain triggers), and/or other factors. The system may extrapolate an appropriate remedial action for each trigger associated with the user. Based on the custom profile 802, the system may, for example, skip scenes containing tornados, summarize scenes containing blood, and offer counseling resources for scenes containing guns (e.g., the counseling resources may be offered based on the extended boundaries 1103A, for example, if the lead-up events to a gun violence scene trigger an increased heart rate in a user, the counseling resources may be offered during the boundaries 1103 to mitigate the user's stress symptoms). As remedial actions are offered, the user may offer feedback regarding filtering and remedial actions, so if the user would prefer a calming scene to replace blood, they may indicate such preferences as shown in FIG. 13.

The profile setup may be as shown in FIG. 5D, and in step 1711, trigger notification options 501F may be displayed. A user may prefer not to be notified of identified triggers, for example, the user may want the remedial actions to be applied to identified triggers without receiving notifications for a seamless viewing experience. Another user may be highly sensitive to all of their designated triggers and may thus prefer to be notified for all identified triggers, which may allow them to anticipate potential stress responses. Yet another user may prefer to receive notification for triggers with specific severities (e.g., the user may wish to be notified of identified guns with severities above a level of 0.50 such as guns laying on a table which may be identified in a video frame, so guns with levels below 0.50, such as gunshots occurring offscreen, may be remediated without notification). In step 1712, if the setup is complete, the process 1700B may end. If the user would like to continue generating stress profiles, then step 1702 may be implemented and the processes 1700A-B may continue. The options designated throughout the user account setup processes 1700A-B may be stored in the databases 700 and 417.

A user may select and/or generate any number of general and/or custom stress profiles. For example, the user may select the “Natural Disaster” stress profile 801B shown in FIG. 8A. The user may be sensitive to other triggers (e.g., guns and blood) and elect to generate the custom stress profile 802 shown in FIG. 8B. Using the steps in the processes 1700A-B described above, the user may configure profiles tailored to any possible trigger. The user may elect to view content filtered according to any one or more of their stress profiles.

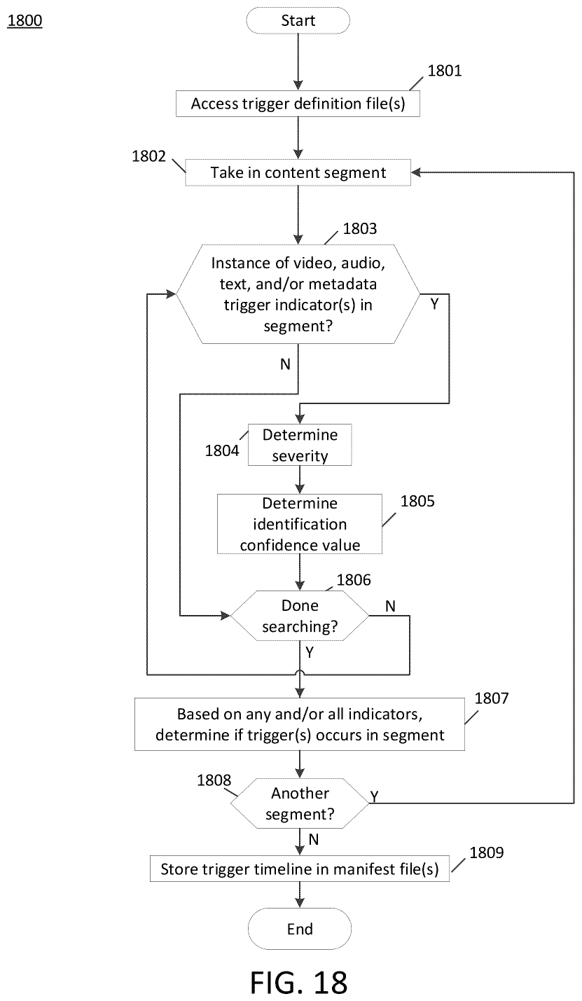

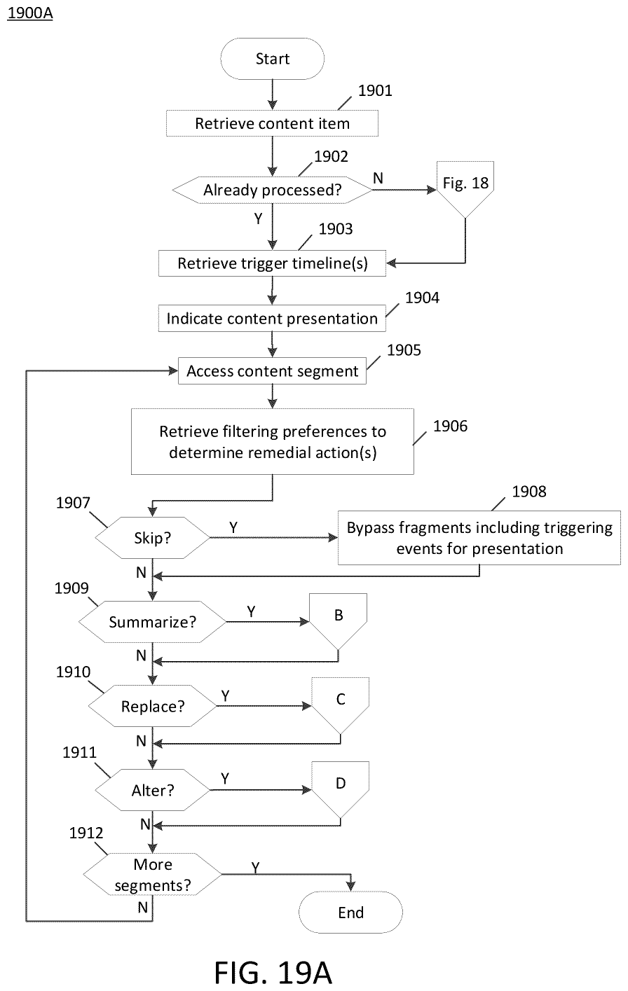

FIG. 18 is a flow chart showing an example process 1800 for identifying trigger events in a content item (e.g., a movie). The content analysis processes 401 may be implemented by the process 1900. In step 1801, trigger definition file(s) may be retrieved to inform the system of how to recognize different kinds of stress indicators. The definition files may be as shown in the database 900A, indicating various kinds of indicators: visual (e.g., images, video, GIFs, lighting profiles, etc.), audio (e.g., sound files, sound patterns, etc.), text (e.g., closed captions, transcripts, etc.), metadata (e.g., manifest files, trigger codes, trigger timestamps, etc.), and/or other kinds, and step 1801 may retrieve information describing the kinds of indicators. For example, if the trigger being searched for is tornados, then step 1801 may comprise retrieving information that has been previously associated with tornados—for example, video image information showing images of tornados, audio patterns for sounds of tornado winds, text data of the words “tornado” and “storm”, and/or metadata describing the presence of storms in a movie (e.g., a metadata stream may include a code “trigger399” denoting tornados, as shown in the entry 901G in FIG. 9A). Trigger indicator data may include reference files and/or file types against which indicators found in the content item may be compared to determine trigger occurrence. Based the definition files, the system may determine, using on the indicators identified in the content item, whether the trigger being searched for occurs in the content item. This determination may be based on a variety of factors, including the number of trigger indicators, the types of trigger indicators, the indicator severity level (e.g., like the levels 910-913 in FIG. 9B), etc.

In step 1802, a content segment may be taken in for processing. The processing may include analysis of various data streams, including the video data, audio data, text data, and/or metadata. Based on the definition files, in step 1803, a determination is made as to whether trigger indicators occur in the content segment. The content analysis processes 401 may be performed on segments of the content item (e.g., scenes of a movie, content fragments in a manifest file, etc.) during the determination step. A sci-fi movie may include scenes of various natural disasters, including tornados. Segments of the movie may be processed to determine whether they contain tornados. A segment may be searched for contemporaneous visual, audio, text, and/or metadata indicators of tornados corresponding to the types of indicators denoted in the trigger indicator database 900A and/or the tornado trigger definition file described above.

In step 1803, data streams associated with the content item (e.g., video data, audio data, text data, metadata, etc.) may be searched for their respective indicators via the content analysis processes 401. Potential triggers in the content item may be analyzed in comparison with the indicators retrieved in step 1801 to determine whether a triggering event actually occurs in the content item. This step may comprise comparing indicators found in the content item with indicators stored in trigger definition files/databases. For example, an image of a tornado may be identified via a video frame in the movie, which may be compared with visual indicator definitions associated with tornados (e.g., “tornado.png”, etc.). Based on this comparison, the system may determine whether this image contains a tornado visual indicator (which may later be referenced in combination with other identified trigger indicators, such as audio and metadata indicators, to determine whether a tornado occurs in the content segment of interest, as described further for step 1807 below).

The trigger entry 901G in FIG. 9A shows various types indicators which may be associated with the trigger tornados. To search a content item for tornados, its associated video data may be analyzed for visual tornado indicators (e.g., compared to images of tornados (e.g., image file “tornado.png,” video of tornados (e.g., video file “tornado.mp4,” etc.). The audio data may be analyzed for audio tornado indicators (e.g., tornado sounds like strong winds in audio file “tornado.mp3”). The text data, which may include transcripts and/or closed captioning data, may be analyzed for text tornado indicators (e.g., spoken words like “tornado”, “storm”, etc. in text file “tornado.csv”). The metadata, which may include manifest file(s) associated with the content item, may be analyzed for metadata indicators (e.g., trigger codes, a tornado alert, etc.). A news program, for example, may be associated with metadata like a tornado alert which may be sent to viewers in affected areas. The tornado alert may be identified as a metadata indicator of a tornado via the content analysis process(es) 401.

Trigger indicators identified in step 1803 may correspond to any number of triggers, for example, the content segment may include visual indicators of tornados and audio indicators of gunshots, etc. When a triggering event is detected, the location of the start and end times may be stored as the trigger's boundaries 1103. If events related to the primary trigger are detected before and/or after the identified start and end times, the boundaries 1103 may be appended with the extended boundaries 1103A indicating lead-up and/or comedown events (e.g., if the primary trigger is assault, then a related event may be an assailant chasing a victim into an alleyway, which may precede an assault scene in the movie). A user sensitive to assault may begin experiencing stress symptoms at events implying assault, not only to explicit images/sounds of assault, so the extension of the boundaries 1103 to 1103A may allow the user to be offered remedial actions sooner, which may decrease their stress reaction to a triggering assault scene.

After an indicator is identified, in step 1804, an indicator level (e.g., a severity level) of the indicator, as shown in the levels 910-913 in FIG. 9B, may be determined. This step may be performed for each indicator detected in the segment. Severity levels may be assigned based on various factors, including the quantity of identified indicators matching the definitions retrieved in step 1801. For example, the movie may include a scene showing a tornado appearing on the horizon, moving closer, and eventually destroying some homes. During analysis of this scene, the system may identify images of the tornado far away and images of the tornado destroying homes, which may be visual trigger indicators. Identified indicators may be compared to the definitions, and based on the quantity of frames matching the definitions, the indicators may be assigned severity levels (e.g., high severity for larger quantities of matching images). For example, the visual tornado indicator comprising images of a distant tornado appearing small on a landscape may be assigned a low severity level (e.g., a level of 0.20). The visual tornado indicator comprising images of a large tornado filled with debris, demolishing several homes, may be assigned a high indicator level (e.g., a level of 0.90). These levels may be assigned based on determinations that the images corresponding to the large tornado filled with debris may match multiple of the trigger indicator definitions (e.g., matching “tornado.png”, “tornado-debris.png”, multiple frames of “tornado.mp4”, etc.), while the images corresponding to the distant tornado may match fewer trigger indicator definitions (e.g., matching “tornado.mpg”, some frames of “tornado.mp4”, etc.).

The determination to perform remedial actions, if any, according to user preferences, may be based on trigger indicator levels. For a user highly sensitive to tornados who elects high filtering, any identified tornados with indicator levels greater than or equal to 0.10 may trigger their preferred remedial actions; for this user, both of the 0.20-level and 0.95-level tornados may trigger remedial actions. For a user slightly sensitive to tornados who elects low filtering, only identified tornados with indicator levels greater than or equal to 0.95 may trigger their preferred remedial actions; for this user, neither of the 0.20-level or 0.95-level tornados may trigger remedial actions. Identification of contemporaneous occurrences of multiple trigger indicators may indicate a higher severity value, for example, a tornado identified via visual, audio, and text indicators (e.g., image “tornado.png”, sound file “tornado.mp3”, dialog file “tornado.csv”, etc.) may be assigned a greater severity value than a tornado identified via a text indicator (e.g., “tornado.csv”) alone, since a user may experience a greater stress reaction to a scene showing an active tornado with people shouting the word “tornado”, for example, than to a scene where characters may be simply discussing tornados.

In step 1805, a trigger identification confidence value may be calculated indicating how confident the system may be that a trigger was accurately identified. This confidence value may be output to the trigger definition file and/or to a trigger timeline associated with the content item (further described below for step 1809). A greater confidence value may be associated with a trigger which is indicated by several types of indicators, for example, a tornado identified via a combination of visual, audio, text, and metadata indicators may be assigned a greater confidence value than a tornado identified via an audio indicator. Contemporaneous occurrences of more than one type of indicator may indicate a trigger with greater confidence and/or accuracy. A tornado alert issued by a local authority (e.g., “tornado_alert.csv”), which may be identified as a metadata indicator, may be assigned a high confidence value (e.g., a level of 1.00), since tornado alerts issued by local authorities may be considered as accurately indicating tornados. A high indicator level may be associated with a higher calculated total severity (as described for FIG. 9B). The metadata indicator may be assigned the 1.00 confidence value based on the tornado alert being unambiguous (e.g., a code for the trigger which may be denoted as a binary value 1.0). The confidence value may be based on a degree to which the identified indicator matches the indicator definitions retrieved in step 1801 (e.g., the quantity of matches, etc.), as described above. If no trigger indicators are identified in step 1803, then the process 1800 may proceed to step 1806. Steps 1804-1805 may be performed for each indicator identified in step 1803. Step 1806 determines whether more of the defined trigger indicators (e.g., triggers defined in the trigger database 900A, the definition files, etc.) have been detected in the content segment. If there are more potential indicators to find, the process may return to step 1803, which may be performed until all defined trigger indicators have been found in the segment. If processing of the segment is complete, then in step 1807, a calculation based on the stored indicators may be performed to determine whether triggers corresponding to the detected indicators occur in the content segment (e.g., the weighting processes 407 may perform the calculation). The calculation may determine whether the trigger indicators detected above are sufficient to be considered an actual trigger based on a total severity level (e.g., the total severity 909 in FIG. 9B), which may depend on some combination of the visual, audio, text, and/or metadata indicator levels assigned to the indicators detected in the segment. To determine whether a trigger occurs, the total severity may be compared to a filtering threshold (e.g., a severity threshold, as shown by the filtering level indicated in FIG. 6), which may be determined by information in general and/or custom stress profiles.

The process 1800 may be executed to process a content item (e.g., a movie), so step 1808 determines whether there are more content segments associated with the content item to be processed. If there are more content segments to be processed, step 1903 1802 may be implemented to take in the next content segment, and the process 1900 may continue for that segment. After the content segments are processed, in step 1809, a trigger timeline (e.g., a stress event schedule) may be generated. The trigger timeline may include information indicating the trigger locations, types, severities, confidence values, etc. determined based on the steps 1801-1808, within the content item. The timeline may indicate fragments of the content item, within the manifest file, that contain the trigger. Trigger codes may denote the locations of identified triggers in the trigger timeline (as shown in 905 of FIG. 9A). This timeline may be stored in the metadata and/or manifest file(s) associated with the content item. The data associated with the timeline may be used as metadata indicators in other processing and/or reprocessing of the content item or its segments.

The timeline may include boundaries indicating approximate start and end times associated with a triggering event detected in the content item. For example, as shown in 914B-D in FIG. 9B, at time 10:43 in the movie the hero draws the gun, at time 11:07 the hero shoots the villain with the gun, and at time 11:09 the villain has a bleeding gunshot wound. These timestamps may indicate the start of the triggering events in the movie. A user with high sensitivity to gun violence may benefit from earlier warning of gun violence and/or later opportunities for remedial actions. Based on the identification of the 0.95-level gun trigger at time 11:07, step 1809 may determine that the drawing of the gun and the bleeding villain may be triggering to the user, so the trigger boundaries 1103 may be appended with extended boundaries 1103A indicating the locations of lead-up and comedown events relating to the primary trigger. As additional content may be processed, trigger identification accuracy may improve as the system (e.g., machine learning models) learns to identify triggers. Information retrieved via feedback from users, as shown in FIG. 13, may indicate accuracy of trigger identification, and the system may use this information in calculating trigger identification confidence values.

FIGS. 19A-D are flow charts showing example processes 1900A-D illustrating the remedial actions offered to users which may alleviate stress symptoms associated with triggers in content. The processes 1900A-D may be implemented when content is presented to users. A content item (e.g., a movie) may be presented to a user who may have a profile configured via the processes 1700A-B. In step 1901, the content item and any associated manifest file(s) may be retrieved. If a movie has already been processed, its associated data may have been analyzed following the steps in the process 1800. The manifest file(s) associated with the movie may include a trigger timeline resulting from the processing. The movie may have been processed as part of batch processing of content. For example, as new content items are acquired (e.g., to be added to content servers such as the content server 106), they may be preprocessed (e.g., processed prior to playback by users) for common types of triggers, which may correspond to the general stress profiles described above.

Step 1902 determines whether the content has already been processed for triggers as described above in FIG. 18 (e.g., as part of batch processing which may include the user's trigger(s)). If the user has previously watched the movie, it may have been previously processed according to at least one of the user's stress profiles. If the content has been previously processed, associated trigger timelines may be retrieved in step 1903. If the content has not been already processed, the process 1800 in FIG. 18 may be implemented. In step 1904, presentation of the content may be indicated (e.g., instructions to begin playback, streaming, accessing unfiltered content, etc. on a user device such as the devices associated with the users 302 may be transmitted). Content items may be presented via segments (e.g., short fragments in manifest files), so in step 1905, a content segment containing a trigger may be retrieved. The process 1800 may be implemented as content segments may be presented to the user, for example, content segments (e.g., content fragments in the manifest file(s)) may be processed according to 1801-1808 and presented to the user. During presentation of a processed content segment, the next segment may be processed according to steps 1802-1807. The steps 1801-1808 may implemented for the full content item selected for presentation to the user. After the content item is processed, then step 1809 may be implemented, generating a trigger timeline to be associated with the content item's manifest file, similar to the result of preprocessing the content item. In step 1906, filtering preferences may be retrieved. Filtering preferences may include custom stress profiles and/or general stress profiles. For a user playing back an action movie, for example, any remedial actions executed may correspond to the user's custom stress profile.

Stress profiles may indicate which remedial action may be offered based on certain triggers. As shown in the interface 1100A in FIG. 11A, the user may receive the notification 1102A indicating that a trigger (e.g., the car crash triggering event 1101) was detected in the movie in a current and/or upcoming scene. More than one remedial action may be offered for the same trigger. For example, a user may wish to have a trigger skipped and a summary of the events in the skipped segments displayed. The implementation of more than one remedial action is indicated by example in the process 1900A via sequential determinations of which remedial action may be offered (e.g., via steps 1907, 1909, 1910, and/or 1911). In step 1907, it may be determined whether the stress profile indicates that the trigger should be skipped. If the stress profile indicates that the trigger should be skipped, then in step 1908, the fragments including the trigger may be bypassed for presentation to the user, and the process 1900A may proceed to step 1909. As shown in FIG. 11B, the user may receive the notification 1102B indicating that the trigger has been skipped. If the trigger should not be skipped, then in step 1909, it may be determined whether the stress profile indicates that the content segment containing the trigger should be summarized. If the segment should be summarized, the process 1900A may proceed to step 1913 of process 1900B in FIG. 19B.

In step 1913, a determination is made as to whether relevant metadata is associated with the content item (e.g., included in the manifest file(s) associated with the content, program listing information, etc.). The metadata may include scene descriptions, content summaries, descriptions of triggers, etc. If relevant metadata is found, then in step 1914, the metadata corresponding to the segments containing the triggering event(s) may be retrieved. In step 1915, a summary may be generated based on the retrieved metadata. Generated summaries may include details indicating information describing other events occurring in the segment containing the trigger. For example, the scene showing the car crash may include other events relevant to the plot of the movie (e.g., whether a character survives the crash), as shown in the summary 1104. In step 1916, the generated summary may be stored for presentation to the user (e.g., in the manifest file(s) with instructions for the location, in the movie, at which the summary may be displayed to the user). The process 1900B may proceed to step 1910.

In step 1913, if no relevant metadata is found, then in step 1917, external sources (e.g., the social media platforms 304, the media sources 306, etc.) may be searched for data corresponding to the identified trigger in the content segment, and step 1918 may determine whether relevant information is found. Another user who previously watched the same movie may have created a social media post relating to the identified trigger (e.g., stating “I can't believe Bobby survived that awful car crash!”). In step 1919, relevant information from the social media post describing the trigger and background events may be retrieved, and the summary 1104 may be generated based on this information. If no relevant information is found via searching external sources, in step 1920, a message indicating that no summary is available may be stored may be stored for presentation to the user (e.g., in the manifest file(s) associated with the movie). The process 1900B may proceed to step 1910.

Step 1910 may determine whether the stress profile indicates that the trigger should be replaced. If the trigger should not be replaced, then the process 1900A may proceed to step 1911. If the segment should be replaced, the process 1900A may proceed to step 1921 of process 1900C in FIG. 19C. In step 1921, a determination is made as to which type of alternate content may replace the trigger. Alternate content may include alternate scenes, calming content, links to counseling resources, and/or advertisements. If the type alternate content selected is an alternate scene, then step 1922 may determine whether alternate scenes are available (e.g., an available alternate scene may include segments stored in the content item's manifest file(s), which may be indicated as an alternate scene relating to certain boundaries such as the boundaries 1103). Alternate scenes may include scenes from edited-for-tv versions of content. If an alternate scene is available, then in step 1923, the fragments containing the alternate scene may be retrieved, and in step 1925 the alternate fragments may be stored for presentation to the user (e.g., in the manifest file(s) associated with the movie with instructions to present these fragments instead of the fragments containing the trigger). The user may be presented with the alternate scene 1105. If no alternate scene is available, then in step 1924, a message indicating that no alternate scene is available may be stored for presentation to the user (e.g., in the manifest file(s) associated with the movie). The process 1900C may proceed to step 1911.

If the type of alternate content selected is calming content, then in step 1926, the calming content may be retrieved. Calming content may comprise nature scenes (e.g., a gentle river as illustrated in FIG. 11E), calming music, guided meditations, user-designated content, and/or other content which may alleviate stress symptoms due to triggers. A user who is sensitive to car crashes may elect to view a scene of a gentle river instead of the portion of the movie containing the car crash. In this case, the fragments containing the trigger may be skipped and the calming scene may be presented instead. In step 1927, fragments corresponding to the calming content may be stored for presentation to the user (e.g., in the manifest file(s) associated with the content item). The process 1900C may proceed to step 1911.

If the type of alternate content selected is counseling resources, in step 1928, information indicating counseling resources may be retrieved. As illustrated in the message 1107 in FIG. 11F, counseling resources may include contact information and/or links to organizations like the National Center for PTSD, links to articles indicating coping strategies (e.g., for stress, anxiety, etc.), and/or other information which may be helpful to a user who may be experiencing stress symptoms. The information retrieved may be tailored to the user, for example, a user whose triggers include domestic violence may be shown information indicating counseling resources for victims of domestic violence, a user who selected the “Veteran” pre-generated profile may be shown information indicating resources for veterans, etc. In step 1929, the information may be stored for presentation to the user with instructions to present the message 1107 instead of the scene containing the triggering. The user may prefer the scene to continue playing in the background with the message 1107 overlaid over the content. The process 1900C may proceed to step 1911.

If the type of alternate content selected is advertisements, then in step 1930, relevant advertisements may be retrieved (e.g., from the ad server 123 and/or other servers). Advertisements may contain events triggering to certain users (e.g., a trailer for an action movie may include a scene with a dramatic, explosive car crash, which may be triggering for a user sensitive to car crashes). The advertisements may have been preprocessed (as discussed above relating to FIG. 19A). Step 1931 determines whether the selected advertisements have already been processed. If they have been preprocessed, then in step 1932, they may be inserted for presentation to the user instead of the scene containing the trigger. If the selected advertisements have not been preprocessed or if the preprocessing does not include the user's trigger (e.g., if car crashes may not be considered a common trigger), the process 1800 may be implemented at least partially for the selected advertisements (e.g., presenting a processed fragment of the advertisement during processing of the next fragment, which is further described below). The filtered advertisements may then be inserted for presentation to the user instead of the scene containing the trigger. The process 1900C may proceed to step 1911.

Step 1911 may determine whether the stress profile indicates that the trigger should be altered. If the trigger should not be altered, then the process 1900A may proceed to step 1912, in which it may be determined whether there are more segments to which the processes 1900A-D may be applied. If there are more segments, the process 1900A may proceed to step 1905 and continue for other segments containing the same and/or other triggers. In step 1911, if it is determined that the segment should be altered, the process 1900A may proceed to step 1933 of process 1900D in FIG. 19D.

Triggering events may be altered via obstruction of areas in the content containing triggers, altering the audio associated with the content segment containing the trigger, and/or altering the video associated with the content segment containing the trigger. More than one type of alteration may be implemented for the same trigger. For example, the user may wish to have a brightness and volume associated with the scene containing the trigger to be decreased. The implementation of more than one alteration is indicated by example in the process 1900D via sequential determinations of which alteration may be offered (e.g., via steps 1933, 1935, and/or 1937).

In step 1933, it may be determined whether obstruction should be implemented. If obstruction is not selected, then the process 1900D may proceed to step 1935. Obstruction may occur via blurring, blocking, and/or otherwise decreasing visibility during the scene containing the trigger. The obstruction may be applied to a portion of the screen showing the trigger or to the whole screen. If the type of alteration selected is obstruction, then in step 1934, a graphic may be generated and instructions for its presentation to the user may be stored in the manifest file(s) associated with the content item. The process 1900D may then proceed to step 1935. The size, location, and/or other properties of the graphic may be determined according to user preferences. For example, the user may wish to only have the portion of the screen showing the car crash to be obstructed, as illustrated by the graphic 1109 in FIG. 11H.

In step 1935, it may be determined whether auditory alteration should be implemented. If auditory alteration is not selected, the process 1900D may proceed to step 1937. A user sensitive to the loud clanging and crashing sounds associated with car crashes may prefer to have the volume muted during car crashes, as illustrated by the volume indicator 1110 in FIG. 11I. If auditory alteration should be implemented, then in step 1936, instructions for altering the audio track associated with the triggering events may be stored in the associated manifest file(s) for presentation to the user. Auditory alterations may include decreasing the volume, muting the volume, and/or otherwise altering auditory properties of the segment containing the trigger. The process 1900D may proceed to step 1937.

In step 1937, it may be determined whether visual alteration should be implemented. If visual alteration is not selected, the process 1900D may proceed to step 1912. A user sensitive to bombs may be triggered by bright flashes associated with explosions and prefer to have the brightness in scenes containing bright flashes decreased, as illustrated in FIG. 11J. If visual alteration should be implemented, then in step 1938, instructions for altering the video containing the triggering events may be stored in the associated manifest file(s) for presentation to the user. Visual alterations may include decreasing the brightness, contrast, resolution, and/or other visual properties of segment containing the trigger. The process 1900D may proceed to step 1912, which is described above.

The processes 1900A-D may be implemented for any segments which contain triggering events. While setting up profiles (e.g., as shown in the processes 1700A-B), users may indicate the type of remedial action to be implemented based on identified triggers. A user may, for example, indicate that gun violence should be skipped and that tornados should be replaced with calming nature scenes. The implementation of various remedial actions may mitigate stress symptoms and support improved mood in users. The remedial actions may be implemented automatically, so users can designate their preferred remedial actions in their user profile and then experience content while avoiding or minimizing the severity of their triggers, which may encourage them to continue viewing it.

FIGS. 20A-B are flow charts showing example operational methods for presenting and remediating content to a user device. A user may select a movie for presentation, so in step 2001, the selected content item may be retrieved. Step 2002 may determine whether the trigger filtering should be applied to the content. The user may be presented with options to filter the movie (e.g., via the filtering button 1503 in FIG. 15B) or to watch the movie without filtering. The user may have designated, in their profile, that trigger filtering should be applied to certain types of content. If the user elects to view the movie without filtering, the process 2000A may proceed to step 2008, which may begin playback. If the user elects to user filtering, then step 2003 may determine whether the user already has any stress profile(s) configured (e.g., by checking the user's account data in the database 700, for example). If not, then the processes 1700A-B may be implemented. If the user already has a stress profile configured, then step 2004 may determine whether the content selected for presentation is unfiltered. If unfiltered content is selected for presentation, then the process 2000A may proceed to step 2011. If the content is not live, then step 2005 determines whether the content is preprocessed. If the content is not preprocessed, then the process 1800 may be at least partially implemented, and in step 2006, the filtered content may be retrieved. The trigger timeline generated by the filtering may be retrieved. The process 1800 may be implemented as content segments may be presented to the user, for example, content segments (e.g., content fragments in the manifest file(s)) may be processed according to 1801-1808 and presented to the user. During presentation of a processed content segment, the next segment may be processed according to steps 1802-1807. The steps 1801-1808 may implemented for the full content item selected for presentation to the user. After the content item is processed, then step 1809 may be implemented, generating a trigger timeline to be associated with the content item's manifest file, similar to the result of preprocessing the content item. If the content is preprocessed, step 2006 may be implemented.

In step 2007, remedial actions indicated by the user (e.g., in their stress profile) may be offered based on the corresponding triggers. For example, a sci-fi movie may include scenes showing explosions, battles, and bleeding injuries. The user may prefer different remedial actions for each of the identified type of trigger (e.g., silencing explosions, replacing battles with guided meditations, and obstructing blood). Instructions indicating the types of remedial action and location in the content in which they may be implemented may be stored, for example, in manifest file(s) associated with the content in step 2007. In step 2008, presentation of the selected content may begin. During content presentation, a user may determine that they have just seen an event that is triggering for them, which may not have been designated as a trigger during profile set up (e.g., stress profile configuration as shown in the processes 1700A-B). The user may mark the location in the content where the trigger occurs via the flag button 1401, as illustrated in FIGS. 14A-C. Step 2009 may determine whether additional triggers may be identified during presentation (e.g., via the flagging options described, via extrapolation based on user heart rate data retrieved via the devices 303, etc.). If new triggers and/or additional occurrences of known triggers are flagged, the user's profile may be updated to include the relevant data in step 2010. The rest of the content being presented and/or other content presented in the future may be filtered according to the triggers flagged triggers, in addition to others selected by the user.

Process 2000B shows a method for presentation and filtering of content that may not have been already filtered, including live content, content that has not been preprocessed, etc. After step 2004 determines that the content selected for presentation has not been filtered (e.g., a live sporting event), then in 2011, options for applying a buffer (e.g., the buffer prompt 1507 in FIG. 15C) may be offered to the user. The user may indicate whether or not a buffer should be applied as well as the length of an applied buffer, which may indicate the time by which presentation of the live content may be delayed to allow for filtering. If the user elects to use a buffer, then in step 2012, delayed presentation of the content may begin according to the length indicated by the user. During the delay, some version of the process 1800 may be implemented in filtering the segment(s) of the content accessible due to the delay. Based on the results of the filtering (e.g., the trigger timeline), then in step 2013, remedial actions corresponding to the user's preferences may be implemented for identified triggers. For example, the user may be a traumatic brain injury (TBI) survivor and may be sensitive to content showing concussions and/or head injuries. If a head injury is identified in the content, the user may prefer to have advertisements displayed instead of the portion showing the head injury. The remedial actions may be implemented as triggers are detected by filtering during the presentation delay.

If the user elects not to use the buffer, then step 2014 may search metadata associated with the content and/or external sources (e.g., the social media platforms 304, the media sources 306, etc.) for information indicating triggers associated with the user. Posts and/or articles associated with the external sources 304 and/or 306 may be searched for information indicating that a player has sustained a head injury during the sporting event. Step 2015 may determine whether information indicating triggers was found. If information was found, the location of the trigger may be marked (e.g., in a trigger timeline) in step 2016, and the relevant remedial action(s) may be applied based on the user's preferences in step 2013. The process 2000B may proceed to step 2009, which determines whether additional triggers may be identified during content presentation. Additional triggers may be identified based on input from the devices 303 (e.g., a smart watch). Another user sensitive to viewing head injuries may not have a stress profile configured. This user may be watching a sporting event when a player sustains a serious head injury. The user's smart watch may detect a rapid increase in heart rate and/or other symptoms of stress or anxiety, which may indicate that the user has viewed a triggering event. The filtering system may learn that this user is sensitive to head injuries and implement remedial actions, presenting the user with a guided meditation to aid in mitigating their symptoms. Having learned that the user exhibits stress responses to head injuries, the system may then implement remedial actions in advance of presentation of head injuries in the future.

If several users are watching a movie when a scene appears containing events triggering for one user, that user may leave the room. One of the devices 303, a camera, for example, may detect the user leaving and the system may implement various remedial actions for the users. The user who left the room may be presented with a guided meditation (e.g., a message indicating the guided meditation may be sent to the user's mobile device). The users remaining in the room may be presented with counseling resources which may indicate how to support a friend experiencing anxiety or stress symptoms. In step 2010, information indicating the newly identified triggers may be stored, which may be used in filtering and providing remedial actions for other content.

Although examples are described above, features and/or steps of those examples may be combined, divided, omitted, rearranged, revised, and/or augmented in any desired manner. Various alterations, modifications, and improvements will readily occur to those skilled in the art. Such alterations, modifications, and improvements are intended to be part of this description, though not expressly stated herein, and are intended to be within the spirit and scope of the disclosure. Accordingly, the foregoing description is by way of example only, and is not limiting.

1. A method comprising:
receiving, by a computing device:
a stress event schedule indicating portions of a content item in which stress events occur; and
a user stress profile indicating a type of stress trigger associated with a user;
causing, based on a comparison of the stress event schedule and the type of stress trigger associated with the user, and during output of the content item to the user:
output of a message indicating that a stress triggering scene has been detected in the content item; and
modified output, of the content item, based on the detected stress triggering scene.

2. The method of claim 1, further comprising:
causing output of an interface indicating a plurality of types of stress trigger events; and
receiving user selection of one or more of the types of stress trigger events indicated in the interface.

3. The method of claim 1, further comprising:
causing output of an interface indicating a plurality of available responses to stress trigger events; and
receiving user selection of one or more of the responses to stress trigger events.

4. The method of claim 1, wherein the modified output comprises:
automatically skipping the detected stress triggering scene;
automatically replacing the detected stress triggering scene with a textual summary of the stress triggering scene;
automatically replacing the detected stress triggering scene with calming content; or
automatically replacing the detected stress triggering scene with information indicating a resource for dealing with the stress triggering scene.

5. The method of claim 1, further comprising generating the user stress profile by causing output of an interface that includes selectable options for:
automatically skipping the detected stress triggering scene;
automatically replacing the detected stress triggering scene with a textual summary of the stress triggering scene;
automatically replacing the detected stress triggering scene with calming content; or
automatically replacing the detected stress triggering scene with information indicating a resource for dealing with the stress triggering scene.

6. The method of claim 1, further comprising receiving user input indicating:
a type of stress event to be avoided; and
a filtering threshold for the type of stress event to be avoided.

7. The method of claim 1, wherein the modified output comprises output, after the detected stress triggering scene is output to the user, of information indicating a resource for dealing with the stress triggering scene.

8. The method of claim 1, wherein the modified output is based on information indicating different remedies for different severities of a type of stress trigger event.

9. A method comprising:
determining, by a computing device, and for at least a first segment of a plurality of segments a content item, an occurrence of a stress triggering event;
determining a severity value associated with the stress triggering event;
determining a confidence value associated with the determining the occurrence of the stress triggering event;
based on the severity value and the confidence value, causing one or more manifest files, associated with the first segment of the content item, to include an indicator of the stress triggering event; and
causing, based on a user stress profile, based on the one or more manifest files, and during output of the content item, modified output of the first segment.

10. The method of claim 9, wherein the causing the one or more manifest files to include the indicator of the stress triggering event comprises causing the one or more manifest files to include an indication of the severity value, and wherein the causing the modified output is further based on comparing the severity value with a threshold indicated in the user stress profile.

11. The method of claim 9, wherein the determining the confidence value comprises determining the confidence value based on a quantity of indicators, in the content item, associated with the stress triggering event.

12. The method of claim 9, wherein the determining the occurrence of the stress triggering event is based on a machine learning model.

13. The method of claim 9, wherein the determining the occurrence of the stress triggering event comprises determining one or more of the following:
one or more auditory indicators of the stress triggering event;
one or more visual indicators of the stress triggering event;
one or more textual indicators of the stress triggering event; or
one or more metadata indicators of the stress triggering event.

14. A method comprising:
causing, by a computing device, output of a listing of a plurality of different types of stress events;
receiving user input indicating that a user wishes to avoid one or more types of stress events selected from the listing;
storing, for the user, a user stress profile based on the user input;
receiving user input indicating a content item selected for output;
determining, based on comparing the selected content item with the one or more types of stress events indicated by the user stress profile, stress events in the content item; and
causing, based on the determined stress events in the content item, modified output of the content item.

15. The method of claim 14, further comprising generating the user stress profile by causing output of a selectable option for automatically skipping a detected stress triggering scene.

16. The method of claim 14, further comprising generating the user stress profile by causing output of a selectable option for automatically replacing a detected stress triggering scene with a textual summary of the stress triggering scene.

17. The method of claim 14, further comprising generating the user stress profile by causing output of a selectable option for automatically replacing a detected stress triggering scene with calming content.

18. The method of claim 14, further comprising generating the user stress profile by causing output of a selectable option for automatically replacing a detected stress triggering scene with information indicating a resource for dealing with the stress triggering scene.

19. The method of claim 14, further comprising causing output of a prompt for a filtering threshold to be used with comparing the one or more types of stress events to stress events in content items.

20. The method of claim 14, wherein the causing of the modified output is based on weighting different trigger indicators differently for a first type of stress event.
